(19) United States
(12) Patent Application Publication
(10) Pub. No.: US 2023/0348902 A1
(43) Pub. Date: Nov. 2, 2023

(54) METHODS AND COMPOSITIONS FOR MAKING ANTIBODY LIBRARIES AND ANTIBODIES ISOLATED FROM THE SAME

(71) Applicant: INTEGRAL MOLECULAR, INC., Philadelphia, PA (US)

(72) Inventors: Trevor A. BARNES, Philadelphia, PA (US); Joseph B. RUCKER, Philadelphia, PA (US); Ross S. CHAMBERS, Philadelphia, PA (US)

(21) Appl. No.: 18/041,161

(22) PCT Filed: Aug. 10, 2021
(86) PCT No.: PCT/US2021/045360

§ 371 Date: Feb. 9, 2023

Related U.S. Application Data
(60) Provisional application No. 63/063,647, filed on Aug. 10, 2020.

(51) Int. Cl. C07K 16/00; C12N 15/10

(52) U.S. Cl. CPC C12N 15/1093; C07K 16/00; C07K 2317/23; C07K 2317/24; C07K 2317/622

(57) ABSTRACT

Embodiments provided herein are directed to the production and identification of antibodies, libraries of antibodies and libraries of antibodies produced in chickens and methods of using the same.

### FIELD

This application relates to methods and compositions for making antibody libraries and antibodies isolated from the same.

### SUMMARY

In some embodiments, methods of producing a population of nucleic acid molecules encoding a chicken complementary determining region (CDR) flanked by two human framework regions (FRs) are provided. In some embodiments, the methods comprise a) amplifying a first population of nucleic acid molecules encoding chicken antibodies with a first primer and a second primer under conditions sufficient to produce the amplified population of nucleic acid molecules encoding the chicken complementary determining region (CDR), wherein: the first primer anneals to a region upstream of a CDR of the chicken antibody, wherein the first primer contains a restriction enzyme recognition site recognized by a restriction enzyme that cleaves at a location that is immediately upstream of the CDR and at a distance away from the recognition site; and the second primer anneals to a region at a distance downstream of the CDR of the chicken antibody. In some embodiments, the restriction site can be upstream of the CDR to produce the first population of nucleic acids. In some embodiments, the restriction site can be downstream of the CDR to produce the first population of nucleic acids.

In some embodiments, methods of producing a library of nucleic acid molecules encoding humanized variable regions of antibodies are provided. In some embodiments, the methods comprise combining i) a first library of nucleic acid molecules encoding a chicken complementary determining region 1 (CDR1) domain, flanked by nucleic acid sequences encoding a human framework region 1 (FR1) and a human framework region 2 (FR2); ii) a second library of nucleic acid sequences encoding a chicken complementary determining region 2 (CDR2) domain, flanked by nucleic acid sequences encoding a human framework region 2 (FR2) and a human framework region 3 (FR3); iii) a third library of nucleic acid sequences encoding a chicken complementary determining region 3 (CDR3) domain, flanked by nucleic acid sequences encoding a human framework region 3 (FR3) and a human framework region 4 (FR4), wherein the nucleic acid molecule encoding the humanized variable regions of antibodies has a formula of:

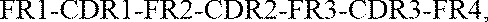

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

In some embodiments, libraries of nucleic acid molecules are provided, wherein the libraries are prepared according to a method provided for herein.

In some embodiments, oligonucleotides that anneal to a region immediately upstream of CDR of a chicken are provided. In some embodiments, an oligonucleotide that anneals to a region immediately upstream of CDR of a chicken antibody, and wherein said oligonucleotide comprises a restriction enzyme recognition site recognized by a restriction enzyme that cleaves at a distance downstream of the recognition site is provided.

In some embodiments, polypeptides encoded by a nucleic acid molecule having a formula of:

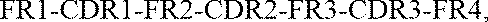

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4,
are provided wherein the nucleic acid molecule is prepared according to a method provided for herein.

In some embodiments, methods of identifying a binding partner to a target are provided. In some embodiments, the methods comprise contacting the target with a library of proteins encoded by a library prepared according to a method as provided for herein.

### BRIEF DESCRIPTION OF FIGURES

FIG. 1 illustrates a non-limiting example of a primer design. The underlined sequence represents the recognition site for the type IIS restriction enzyme AcuI.

FIG. 2 illustrates a non-limiting process for extracting a CDR from a chicken antibody and embedding the CDR in flanking human framework regions.

FIG. 3 illustrates monoclonal scFv binding from an antibody produced according to embodiments provided for herein. Target binding for 91 affinity-matured scFv clones are illustrated. These scFv extracts were tested against their target antigen and a second unrelated membrane protein as control. The bar graph depict the binding of the scFv extracts to the target antigen. The dotted line represents the 20-fold signal:background ratio (target binding). >50% of clones showed a target binding 20-times higher than the binding to the unrelated protein.

### DETAILED DESCRIPTION

As used herein and in the appended claims, the singular forms “a”, “an” and “the” include plural reference unless the context clearly dictates otherwise.

As used herein, the terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

Embodiments provided herein are directed to, in part, methods producing libraries of nucleic acid molecules that encode for CDRs. These CDRs can be derived from, for example, antibodies produced in chickens. In some embodiments, the libraries comprise nucleic acid molecules that encode for a polypeptide that comprises three CDRs, each CDR derived from a chicken antibody.

For example, in some embodiments, methods of producing a population of nucleic acid molecules encoding a chicken complementary determining region (CDR) flanked by two human framework regions (FRs) are provided.

In some embodiments,, the method comprising: a) amplifying a first population of nucleic acid molecules encoding chicken antibodies with a first primer and a second primer under conditions sufficient to produce the amplified population of nucleic acid molecules encoding the chicken complementary determining region (CDR), wherein: the first primer anneals to a region upstream of a CDR of the chicken antibody, wherein the first primer contains a restriction enzyme recognition site recognized by a restriction enzyme that cleaves at a location that is immediately upstream of the CDR and at a distance away from the recognition site; and the second primer anneals to a region at a distance downstream of the CDR of the chicken antibody. In some embodiments, the cleavage that is immediately upstream is about 1, 2, 3, 4, or 5 nucleotides of the CDR boundary. In preferred embodiments, the cleavage that is immediately upstream is about 1, 2, or 3 nucleotides of the CDR boundary. In some embodiments, the second primer anneals to a region at a distance no greater than the length of the mRNA transcript downstream of the CDR of the chicken antibody.

In some embodiments, the method further comprises digesting the amplified population of nucleic acid molecules with said restriction enzyme to produce a 5′ overhang immediately (e.g., 1, 2, 3, or 4 nucleotide bases) upstream of the sequence encoding the CDR to produce a digestion product. In preferred embodiments, the 5′ overhang that is immediately downstream of the sequence encoding the CDR is 1, 2, or 3 nucleotides of the CDR boundary. In some embodiments, the junction sites/overhang can also be a residue in the CDR. For example the 5′ junction sites for CDR-L3 and CDR-H3 are the cysteine upstream of the CDR. In some embodiments, this is considered part of the CDR depending on the CDR naming convention. Therefore, in some embodiments, the overhang is 1 (-1) or 2 (-2) nucleotides outside of the CDR boundary.

In some embodiments, the methods comprise preparing a first ligation product by ligating said digestion product to a nucleic acid sequence encoding a first FR of a human antibody, wherein said first FR of a human antibody comprises an overhang region at its 3′-end compatible with the overhang region of said digestion product so that the first FR is ligated to the upstream of the first digestion product.

In some embodiments, the methods further comprising preparing a second population of amplified nucleic acid molecules with a third primer and a fourth primer under conditions sufficient to produce the second amplified population of nucleic acid molecules encoding the chicken CDR, wherein: the third primer anneals to a region immediately downstream of the nucleic acid sequence encoding the CDR present in the first ligation product, wherein the third primer contains a restriction enzyme recognition site recognized by a restriction enzyme that cleaves at a location that is immediately downstream of the CDR and at a distance away from the recognition site; and the fourth primer anneals to a portion of the nucleic acid molecule encoding the first FR present in the first ligation product at a distance upstream of the CDR in the ligation product. In some embodiments, the restriction enzyme cleavage site that is immediately downstream is about 1, 2, 3, 4, or 5 nucleotides of the CDR boundary. In preferred embodiments, the restriction enzyme cleavage site that is immediately downstream is about 1, 2, or 3 nucleotides of the CDR boundary. In some embodiments, the fourth primer anneals to a portion of the nucleic acid molecule encoding the first FR present in the first ligation product at a distance upstream of the CDR in the ligation product, wherein the distance is determined by the length of the first FR. In some embodiments, the first FR is FR1, FR2, or FR3.

In some embodiments, the method comprises digesting the second amplified population of nucleic acid molecules with said restriction enzyme to produce a 5′ overhang immediately downstream of the sequence encoding the CDR to produce a second digestion product. In some embodiments, the 5′ overhang that is immediately downstream of the sequence encoding the CDR is about 1, 2, 3, 4, or 5 nucleotides of the CDR boundary. In preferred embodiments, the 5′ overhang that is immediately downstream of the sequence encoding the CDR is 1, 2, or 3 nucleotides of the CDR boundary.

In some embodiments, the method comprises ligating the second digestion product to a nucleic acid sequence encoding a second FR of a human antibody to produce the second ligation product, wherein said second FR comprises an overhang region at its 5′-end compatible with the overhang region of said second digestion product, so that the second FR is ligated to the downstream of the second digestion product.

In some embodiments, the method comprises amplifying the second ligation product with a first FW primer that anneals to the first FR of the human antibody and a second primer that anneals to the second FR of the human antibody under conditions sufficient to produce the population of nucleic acid molecules encoding the CDR flanked by two FRs of a human antibody.

In some embodiments, the CDR is a chicken CDR1, CDR2, or CDR3. In some embodiments, the framework regions (FR) are human FR1, FR2, FR3, or FR4. In some embodiments, the first FR is human FR1 and the second FR is human FR2 . In some embodiments, the first FR is human FR2 and the second FR is human FR3. In some embodiments, the first FR is human FR3 and the second FR is human FR4.

The restriction enzyme can be any suitable restriction enzyme. In some embodiments, the restriction enzyme is a Type IIS enzyme. In some embodiments, the Type IIS enzyme is AcuI, BpmI, BpuEI, BsgI, MmeI, or NmeAllI. In some embodiments, the Type IIS enzyme is AcuI, AlwI, Alw26I, BaeI, BbsI, BbsI-HF, BbvI, BccI, BceAI, BcgI, BciVI, BcoDI, BfuAI, BmrI, BmsI, BpiI, BpmI, BpuEI, BsaI, BsaI-HF®v2 (Type IIS restriction enzyme that cuts at a BsaI site), BsaXI, BseRI, BseGI, BsgI, BsmAI, BsmBI, BsmBI-v2, BsmFI, BsmI, BspCNI, BspMI, BspQI, BsrDI, BsrI, BtgZI, BtsCI, BtsI-v2, BtsIMutI, CspCI, EarI, EciI, Eco31I, Esp3I, FauI, FokI, HgaI, HphI, HpyAV, LguI, MboII, MlyI, MmeI, MnlI, Mva1269I, NmeAIII, PleI, SapI, or SfaNI.

In some embodiments, the method further comprises producing a nucleic acid encoding a protein comprising a chicken CDR1, a chicken CDR2, and a chicken CDR3, each CDR flanked by a human framework region. In some embodiments, the step of producing the nucleic acid encoding a protein comprising a chicken CDR1, a chicken CDR2, and a chicken CDR3, each CDR flanked by a human framework region comprises performing overlapping PCR of a first, second, and third ligation product produced according to the methods provided herein, wherein: the first ligation product comprises a nucleic acid molecule encoding a chicken CDR1 flanked by a human framework region; the second ligation product comprises a nucleic acid molecule encoding a chicken CDR2 flanked by a human framework region; and the third ligation product comprises a nucleic acid molecule encoding a chicken CDR3 flanked by a human framework region.

In some embodiments, the step of producing the nucleic acid encoding a protein comprising a chicken CDR1, a chicken CDR2, and a chicken CDR3, each CDR flanked by a human framework region comprises ligating a first, second, and third ligation product produced according to the methods provided herein, wherein: the first ligation product comprises a nucleic acid molecule encoding a chicken CDR1 flanked by a human framework region; the second ligation product comprises a nucleic acid molecule encoding a chicken CDR2 flanked by a human framework region; and the third ligation product comprises a nucleic acid molecule encoding a chicken CDR3 flanked by a human framework region.

In some embodiments, the nucleic acid molecule encoding a protein comprising a chicken CDR1, a chicken CDR2, and a chicken CDR3 has a formula of:

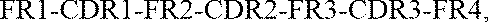

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

In some embodiments, the nucleic acid molecule encoding a protein comprising a chicken CDR1, a chicken CDR2, and a chicken CDR3 encodes an antibody variable region.

In some embodiments, the first primer and third primer comprise a nucleic acid sequence comprising the formula:

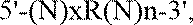

wherein,
R is the recognition sequence;
N is any nucleic acid base, such as naturally occurring, non-naturally occurring, or degenerate nucleic acid base;
x is 0-11; and
n is 1-21.

In some embodiments, R is CTGAAG, CGATC, ACNNNNGTAYC, GAAGAC, GCAGC, CCATC, ACGGC, CGANNNNNNTGC, GTATCC, GTCTC, ACCTGC, ACTGGG, CTGGAG, CTTGAG, GGTCTC, ACNNNNNCTCC, GAGGAG, GTGCAG, GTCTC, CGTCTC, GGGAC, GAATGC, CTCAG, ACCTGC, GCTCTTC, GCAATG, ACTGG, GCGATG, GGATG, GCAGTG, CAGTG, CAANNNNNGTGG, CTCTTC, GGCGGA, CGTCTC, CCCGC, GGATG, GACGC, GGTGA, CCTTC, GAAGA, GAGTC, TCCRAC, CCTC, GCCGAG, GAGTC, GCTCTTC, or GCATC. the formula 5′—R(N)n—3′, wherein R is the recognition sequence, N is any nucleic acid base, and n=1-21. In some embodiments N is any nucleic acid base, such as naturally occurring, non-naturally occurring, or degenerate nucleic acid base. In some embodiments, N is a naturally occurring nucleic acid base, such as C, G, A, T, or U. In some embodiments, the non-naturally occurring nucleic acid base stabilizes the DNA duplex. In some embodiments, the non-naturally occurring nucleic acid base is AP-dC, 2-Aminoadenine, 5-methylcystosine, C(5)-propynylcystosine, or C(5)-propynyluracil. In some embodiments, the degenerate nucleic acid base is K, M, R, Y, S, W, B, D or H. In some embodiments, K is G or T/U. In some embodiments, M is A or C. In some embodiments, R is A or G. In some embodiments Y is C or T/U. In some embodiments, S is C or G. In some embodiments, W is A or T/U. In some embodiments, B is C or G or T/U. In some embodiments, D is A or G or T/U. In some embodiments H is A or C or T/U. In some embodiments, the first primer and third primer comprise a nucleic acid sequence comprising a sequence of 5′-CTGAAGNNNNNNNNNNNNNNNN-3′ (SEQ ID NO: 45).In some embodiments, the first primer and third primer comprise a nucleic acid sequence comprising a sequence of 5′-CGATCNNNN-3′ (SEQ ID NO: 46). In some embodiments, the first primer and third primer comprise a nucleic acid sequence comprising a sequence of 5′-NNNNNNNNNNACNNNNGTAYCNNNNNNNNNNNN-3′ (SEQ ID NO: 47). In some embodiments, the first primer and third primer comprise a nucleic acid sequence comprising a sequence of 5′-GAAGACNN-3′ (SEQ ID NO: 48). In some embodiments, the first primer and third primer comprise a nucleic acid sequence comprising a sequence of 5′-GCAGCNNNNNNNN-3′ (SEQ ID NO: 49). In some embodiments, the first primer and third primer comprise a nucleic acid sequence comprising a sequence of 5′-CCATCNNNN-3′ (SEQ ID NO: 50). In some embodiments, the first primer and third primer comprise a nucleic acid sequence comprising a sequence of 5′-ACGGCNNNNNNNNNNNN-3′ (SEQ ID NO: 51). In some embodiments, the first primer and third primer comprise a nucleic acid sequence comprising a sequence of 5′-NNNNNNNNNNCGANNNNNNTGCNNNNNNNNNNNN-3′ (SEQ ID NO: 52). In some embodiments, the first primer and third primer comprise a nucleic acid sequence comprising a sequence of 5′-GTATCCNNNNNN-3′ (SEQ ID NO: 53). In some embodiments, the first primer and third primer comprise a nucleic acid sequence comprising a sequence of 5′-GTCTCN-3′ (SEQ ID NO: 49). In some embodiments, the first primer and third primer comprise a nucleic acid sequence comprising a sequence of 5′-ACCTGCNNNN-3′ (SEQ ID NO: 55). In some embodiments, the first primer and third primer comprise a nucleic acid sequence comprising a sequence of 5′-ACTGGGNNNNN-3′ (SEQ ID NO: 56). In some embodiments, the first primer and third primer comprise a nucleic acid sequence comprising a sequence of 5′ -CTGGAGNNNNNNNNNNNNNNNN-3′ (SEQ ID NO: 57). In some embodiments, the first primer and third primer comprise a nucleic acid sequence comprising a sequence of 5′-CTTGAGNNNNNNNNNNNNNNNN-3′ (SEQ ID NO: 58). In some embodiments, the first primer and third primer comprise a nucleic acid sequence comprising a sequence of 5′-GGTCTCN-3′ (SEQ ID NO: 59). In some embodiments, the first primer and third primer comprise a nucleic acid sequence comprising a sequence of 5′-NNNNNNNNNACNNNNNCTCCNNNNNNNNNN-3′ (SEQ ID NO: 60). In some embodiments, the first primer and third primer comprise a nucleic acid sequence comprising a sequence of 5′-GAGGAGNNNNNNNNNN-3′ (SEQ ID NO: 61). In some embodiments, the first primer and third primer comprise a nucleic acid sequence comprising a sequence of 5′-GTGCAGNNNNNNNNNNNNNNNN-3′ (SEQ ID NO: 62). In some embodiments, the first primer and third primer comprise a nucleic acid sequence comprising a sequence of 5′-GTCTCN-3′ (SEQ ID NO: 49). In some embodiments, the first primer and third primer comprise a nucleic acid sequence comprising a sequence of 5′-CGTCTCN-3′ (SEQ ID NO: 63). In some embodiments, the first primer and third primer comprise a nucleic acid sequence comprising a sequence of 5′-GGGACNNNNNNNNNN-3′ (SEQ ID NO: 64). In some embodiments, the first primer and third primer comprise a nucleic acid sequence comprising a sequence of 5′-GAATGCN-3′ (SEQ ID NO: 65). In some embodiments, the first primer and third primer comprise a nucleic acid sequence comprising a sequence of 5′-CTCAGNNNNNNNNN-3′ (SEQ ID NO: 66). In some embodiments, the first primer and third primer comprise a nucleic acid sequence comprising a sequence of 5′-ACCTGCNNNN-3′. In some embodiments, the first primer and third primer comprise a nucleic acid sequence comprising a sequence of 5′-GCTCTTCN-3′ (SEQ ID NO: 67). In some embodiments, the first primer and third primer comprise a nucleic acid sequence comprising a sequence of 5′-GCAATGNN-3′ (SEQ ID NO: 68). In some embodiments, the first primer and third primer comprise a nucleic acid sequence comprising a sequence of 5′-ACTGGN-3′. In some embodiments, the first primer and third primer comprise a nucleic acid sequence comprising a sequence of 5′-GCGATGNNNNNNNNNN-3′. In some embodiments, the first primer and third primer comprise a nucleic acid sequence comprising a sequence of 5′-GGATGNN-3′. In some embodiments, the first primer and third primer comprise a nucleic acid sequence comprising a sequence of 5′-GCAGTGNN-3′. In some embodiments, the first primer and third primer comprise a nucleic acid sequence comprising a sequence of 5′-CAGTGNN-3′. In some embodiments, the first primer and third primer comprise a nucleic acid sequence comprising a sequence of 5′-NNNNNNNNNNNCAANNNNNGTGGNNNNNNNNNNNN-3′. In some embodiments, the first primer and third primer comprise a nucleic acid sequence comprising a sequence of 5′-CTCTTCN-3′. In some embodiments, the first primer and third primer comprise a nucleic acid sequence comprising a sequence of 5′-GGCGGANNNNNNNNNNN-3′. In some embodiments, the first primer and third primer comprise a nucleic acid sequence comprising a sequence of 5′-CGTCTCN-3′. In some embodiments, the first primer and third primer comprise a nucleic acid sequence comprising a sequence of 5′-CCCGCNNNN-3′. In some embodiments, the first primer and third primer comprise a nucleic acid sequence comprising a sequence of 5′-GGATGNNNNNNNNN-3′. In some embodiments, the first primer and third primer comprise a nucleic acid sequence comprising a sequence of 5′-GACGCNNNNN-3′. In some embodiments, the first primer and third primer comprise a nucleic acid sequence comprising a sequence of 5′-GGTGANNNNNNNN-3′. In some embodiments, the first primer and third primer comprise a nucleic acid sequence comprising a sequence of 5′-CCTTCNNNNNN-3′. In some embodiments, the first primer and third primer comprise a nucleic acid sequence comprising a sequence of 5′-GAAGANNNNNNNN-3′. In some embodiments, the first primer and third primer comprise a nucleic acid sequence comprising a sequence of 5′-GAGTCNNNNN-3′. In some embodiments, the first primer and third primer comprise a nucleic acid sequence comprising a sequence of 5′-TCCRACNNNNNNNNNNNNNNNNNNNN-3′. In some embodiments, the first primer and third primer comprise a nucleic acid sequence comprising a sequence of 5′-CCTCNNNNNNN-3′. In some embodiments, the first primer and third primer comprise a nucleic acid sequence comprising a sequence of 5′-GCCGAGNNNNNNNNNNNNNNNNNNNNN-3′. In some embodiments, the first primer and third primer comprise a nucleic acid sequence comprising a sequence of 5′-GAGTCNNNN-3′. In some embodiments, the first primer and third primer comprise a nucleic acid sequence comprising a sequence of 5′-GCTCTTCN-3′. In some embodiments, the first primer and third primer comprise a nucleic acid sequence comprising a sequence of 5′-GCATCNNNNN-3′.

Also provided herein are methods of producing a library of nucleic acid molecules encoding humanized variable regions of antibodies. In some embodiments, the methods comprise combining: i) a first library of nucleic acid molecules encoding a chicken complementary determining region 1 (CDR1) domain, flanked by nucleic acid sequences encoding a human framework region 1 (FR1) and a human framework region 2 (FR2); ii) a second library of nucleic acid sequences encoding a chicken complementary determining region 2 (CDR2) domain, flanked by nucleic acid sequences encoding a human framework region 2 (FR2) and a human framework region 3 (FR3); iii) a third library of nucleic acid sequences encoding a chicken complementary determining region 3 (CDR3) domain, flanked by nucleic acid sequences encoding a human framework region 3 (FR3) and a human framework region 4 (FR4),wherein the nucleic acid molecule encoding the humanized variable regions of antibodies has a formula of:

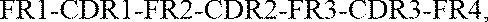

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

In some embodiments, the combining comprises ligating the first, second, and third libraries of nucleic acid molecules to produce the nucleic acid molecule encoding the humanized variable regions of antibodies. In some embodiments, the combining comprises performing overlapping PCR with the first, second, and third libraries of nucleic acid molecules to produce the nucleic acid molecule encoding the humanized variable regions of antibodies. In some embodiments, this is done consecutively.

Also provided herein are libraries of nucleic acid molecules prepared according to any one of the methods provided for herein. In some embodiments, the library comprises a population of nucleic acid molecules, a plurality of nucleic acid molecules of the population encodes a polypeptide having a formula of:

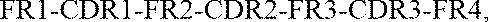

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

In some embodiments, the library comprises at least two nucleic acid molecules present in the library that encode different polypeptides.

In some embodiments, oligonucleotides are provided that anneal to a region immediately upstream of CDR of a chicken antibody, and wherein said oligonucleotide comprises a restriction enzyme recognition site recognized by a restriction enzyme that cleaves at a distance downstream of the recognition site. In some embodiments, the oligonucleotide comprises the sequence of nucleic acid sequence comprising the formula:

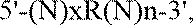

wherein,
R is the recognition sequence;
N is any nucleic acid base, such as naturally occurring, non-naturally occurring, or degenerate nucleic acid base;
x is 0-11; and
n is 1-21.
In some embodiments, R is CTGAAG, CGATC, ACNNNNGTAYC, GAAGAC, GCAGC, CCATC, ACGGC, CGANNNNNNTGC, GTATCC, GTCTC, ACCTGC, ACTGGG, CTGGAG, CTTGAG, GGTCTC, ACNNNNNCTCC, GAGGAG, GTGCAG, GTCTC, CGTCTC, GGGAC, GAATGC, CTCAG, ACCTGC, GCTCTTC, GCAATG, ACTGG, GCGATG, GGATG, GCAGTG, CAGTG, CAANNNNNGTGG, CTCTTC, GGCGGA, CGTCTC, CCCGC, GGATG, GACGC, GGTGA, CCTTC, GAAGA, GAGTC, TCCRAC, CCTC, GCCGAG, GAGTC, GCTCTTC, or GCATC. In some embodiments N is any nucleic acid base, such as naturally occurring, non-naturally occurring, or degenerate nucleic acid base. In some embodiments, N is a naturally occurring nucleic acid base, such as C, G, A, T, or U. In some embodiments, the non-naturally occurring nucleic acid base stabilizes the DNA duplex. In some embodiments, the non-naturally occurring nucleic acid base is AP-dC, 2-Aminoadenine, 5-methylcystosine, C(5)-propynylcystosine, or C(5)-propynyluracil. In some embodiments, the degenerate nucleic acid base is K, M, R, Y, S, W, B, D or H. In some embodiments, K is G or T/U. In some embodiments, M is A or C. In some embodiments, R is A or G. In some embodiments Y is C or T/U. In some embodiments, S is C or G. In some embodiments, W is A or T/U. In some embodiments, B is C or G or T/U. In some embodiments, D is A or G or T/U. In some embodiments H is A or C or T/U. In some embodiments, the oligonucleotide comprises a nucleic acid sequence comprising a sequence of 5′-CTGAAGNNNNNNNNNNNNNNNN-3′ (SEQ ID NO: 45). In some embodiments, the oligonucleotide comprises a nucleic acid sequence comprising a sequence of 5′-CGATCNNNN-3′ (SEQ ID NO: 46). In some embodiments, the oligonucleotide comprises a nucleic acid sequence comprising a sequence of 5′-NNNNNNNNNNACNNNNGTAYCNNNNNNNNNNNN-3′ (SEQ ID NO: 47). In some embodiments, the oligonucleotide comprises a nucleic acid sequence comprising a sequence of 5′-GAAGACNN-3′ (SEQ ID NO: 48). In some embodiments, the oligonucleotide comprises a nucleic acid sequence comprising a sequence of 5′-GCAGCNNNNNNNN-3′ (SEQ ID NO: 49). In some embodiments, the oligonucleotide comprises a nucleic acid sequence comprising a sequence of 5′-CCATCNNNN-3′ (SEQ ID NO: 50). In some embodiments, the oligonucleotide comprises a nucleic acid sequence comprising a sequence of 5′-ACGGCNNNNNNNNNNNN-3′ (SEQ ID NO: 51). In some embodiments, the oligonucleotide comprises a nucleic acid sequence comprising a sequence of 5′-NNNNNNNNNNCGANNNNNNTGCNNNNNNNNNNNN-3′ (SEQ ID NO: 52). In some embodiments,the oligonucleotide comprises a nucleic acid sequence comprising a sequence of 5′-GTATCCNNNNNN-3′ (SEQ ID NO: 53). In some embodiments, the oligonucleotide comprises a nucleic acid sequence comprising a sequence of 5′-GTCTCN-3′ (SEQ ID NO: 49). In some embodiments, the oligonucleotide comprises a nucleic acid sequence comprising a sequence of 5′-ACCTGCNNNN-3′. In some embodiments, the oligonucleotide comprises a nucleic acid sequence comprising a sequence of 5′-ACTGGGNNNNN-3′. In some embodiments, the oligonucleotide comprises a nucleic acid sequence comprising a sequence of 5′-CTGGAGNNNNNNNNNNNNNNNN-3′. In some embodiments, the oligonucleotide comprises a nucleic acid sequence comprising a sequence of 5′-CTTGAGNNNNNNNNNNNNNNNN-3′. In some embodiments, the oligonucleotide comprises a nucleic acid sequence comprising a sequence of 5′-GGTCTCN-3′. In some embodiments, the oligonucleotide comprises a nucleic acid sequence comprising a sequence of 5′-NNNNNNNNNACNNNNNCTCCNNNNNNNNNN-3′. In some embodiments, the oligonucleotide comprises a nucleic acid sequence comprising a sequence of 5′-GAGGAGNNNNNNNNNN-3′. In some embodiments, the oligonucleotide comprises a nucleic acid sequence comprising a sequence of 5′-GTGCAGNNNNNNNNNNNNNNNN-3′ (SEQ ID NO: 62). In some embodiments, the oligonucleotide comprises a nucleic acid sequence comprising a sequence of 5′-GTCTCN-3′ (SEQ ID NO: 49). In some embodiments, the oligonucleotide comprises a nucleic acid sequence comprising a sequence of 5′-CGTCTCN-3′. In some embodiments, the oligonucleotide comprises a nucleic acid sequence comprising a sequence of 5′-GGGACNNNNNNNNNN-3. In some embodiments, the oligonucleotide comprises a nucleic acid sequence comprising a sequence of 5′-GAATGCN-3′. In some embodiments, the oligonucleotide comprises a nucleic acid sequence comprising a sequence of 5′-CTCAGNNNNNNNNN-3′. In some embodiments, the oligonucleotide comprises a nucleic acid sequence comprising a sequence of 5′-ACCTGCNNNN-3′. In some embodiments, the oligonucleotide comprises a nucleic acid sequence comprising a sequence of 5′-GCTCTTCN-3′. In some embodiments, the oligonucleotide comprises a nucleic acid sequence comprising a sequence of 5′-GCAATGNN-3′. In some embodiments, the oligonucleotide comprises a nucleic acid sequence comprising a sequence of 5′-ACTGGN-3′ (SEQ ID NO: 69). In some embodiments, the oligonucleotide comprises a nucleic acid sequence comprising a sequence of 5′-GCGATGNNNNNNNNNN-3′ (SEQ ID NO: 70). In some embodiments, the oligonucleotide comprises a nucleic acid sequence comprising a sequence of 5′-GGATGNN-3′ (SEQ ID NO: 71). In some embodiments, the oligonucleotide comprises a nucleic acid sequence comprising a sequence of 5′-GCAGTGNN-3′ (SEQ ID NO: 72). In some embodiments, the oligonucleotide comprisesa nucleic acid sequence comprising a sequence of 5′-CAGTGNN-3′. In some embodiments, the oligonucleotide comprises a nucleic acid sequence comprising a sequence of 5′-NNNNNNNNNNNCAANNNNNGTGGNNNNNNNNNNNN-3′ (SEQ ID NO: 73). In some embodiments, the oligonucleotide comprises a nucleic acid sequence comprising a sequence of 5′-CTCTTCN-3′. In some embodiments, the oligonucleotide comprises a nucleic acid sequence comprising a sequence of 5′-GGCGGANNNNNNNNNNN-3′ (SEQ ID NO: 74). In some embodiments, the oligonucleotide comprises a nucleic acid sequence comprising a sequence of 5′-CGTCTCN-3′. In some embodiments, the oligonucleotide comprises a nucleic acid sequence comprising a sequence of 5′-CCCGCNNNN-3′ (SEQ ID NO: 75). In some embodiments, the oligonucleotide comprises a nucleic acid sequence comprising a sequence of 5′-GGATGNNNNNNNNN-3′ (SEQ ID NO: 76). In some embodiments, the oligonucleotide comprises a nucleic acid sequence comprising a sequence of 5′-GACGCNNNNN-3′ (SEQ ID NO: 77). In some embodiments, the oligonucleotide comprises a nucleic acid sequence comprising a sequence of 5′-GGTGANNNNNNNN-3′ (SEQ ID NO: 78). In some embodiments, the oligonucleotide comprises a nucleic acid sequence comprising a sequence of 5′-CCTTCNNNNNN-3′ (SEQ ID NO: 79). In some embodiments, the oligonucleotide comprises a nucleic acid sequence comprising a sequence of 5′-GAAGANNNNNNNN-3′ (SEQ ID NO: 80). In some embodiments, the oligonucleotide comprises a nucleic acid sequence comprising a sequence of 5′-GAGTCNNNNN-3′ (SEQ ID NO: 81). In some embodiments, the oligonucleotide comprises a nucleic acid sequence comprising a sequence of 5′-TCCRACNNNNNNNNNNNNNNNNNNNN-3′ (SEQ ID NO: 82). In some embodiments, the oligonucleotide comprises a nucleic acid sequence comprising a sequence of 5′-CCTCNNNNNNN-3′ (SEQ ID NO: 83). In some embodiments, the oligonucleotide comprises a nucleic acid sequence comprising a sequence of 5′-GCCGAGNNNNNNNNNNNNNNNNNNNNN-3′ (SEQ ID NO: 84). In some embodiments, the oligonucleotide comprises a nucleic acid sequence comprising a sequence of 5′-GAGTCNNNN-3′. In some embodiments, the oligonucleotide comprises a nucleic acid sequence comprising a sequence of 5′-GCTCTTCN-3′. In some embodiments, the oligonucleotide comprises a nucleic acid sequence comprising a sequence of 5′-GCATCNNNNN-3′ (SEQ ID NO: 85). In some embodiments, an oligonucleotide is as provided or described herein.

In some embodiments, polypeptides encoded by a nucleic acid molecule having a formula of:

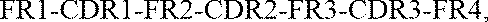

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4,
are provided. In some embodiments, the nucleic acid molecule is prepared according to any method as provided for herein.

In some embodiments, a primer set used to create the library, such as the human framework acceptor fragments comprise a sequence of SEQ ID NO: 2 or a sequence that is at least 90, 91, 92, 93, 94, 95, 96, 97, 98, or 99% identical of SEQ ID NO: 2. In some embodiments, a primer set used to create the library, such as the human framework acceptor fragments comprise a sequence of SEQ ID NO: 3 or a sequence that is at least 90, 91, 92, 93, 94, 95, 96, 97, 98, or 99% identical of SEQ ID NO: 3. In some embodiments, a primer set used to create the library, such as the human framework acceptor fragments comprise a sequence of SEQ ID NO: 4 or a sequence that is at least 90, 91, 92, 93, 94, 95, 96, 97, 98, or 99% identical of SEQ ID NO: 4. In some embodiments, a primer set used to create the library, such as the human framework acceptor fragments comprise a sequence of SEQ ID NO: 5 or a sequence that is at least 90, 91, 92, 93, 94, 95, 96, 97, 98, or 99% identical of SEQ ID NO: 5. In some embodiments, a primer set used to create the library, such as the human framework acceptor fragments comprise a sequence of SEQ ID NO: 43 or a sequence that is at least 90, 91, 92, 93, 94, 95, 96, 97, 98, or 99% identical of SEQ ID NO: 43. In some embodiments, a primer set used to create the library, such as the human framework acceptor fragments comprise a sequence of SEQ ID NO: 6 or a sequence that is at least 90, 91, 92, 93, 94, 95, 96, 97, 98, or 99% identical of SEQ ID NO: 6. In some embodiments, a primer set used to create the library, such as the human framework acceptor fragments comprise a sequence of SEQ ID NO: 7 or a sequence that is at least 90, 91, 92, 93, 94, 95, 96, 97, 98, or 99% identical of SEQ ID NO: 7. In some embodiments, a primer set used to create the library, such as the human framework acceptor fragments comprise a sequence of SEQ ID NO: 8 or a sequence that is at least 90, 91, 92, 93, 94, 95, 96, 97, 98, or 99% identical of SEQ ID NO: 8. In some embodiments, a primer set used to create the library, such as the human framework acceptor fragments comprise a sequence of SEQ ID NO: 9 or a sequence that is at least 90, 91, 92, 93, 94, 95, 96, 97, 98, or 99% identical of SEQ ID NO: 9.

In some embodiments, the primer set comprises a sequence of SEQ ID NO: 2 and SEQ ID NO: 3 or a sequence that is at least 90, 91, 92, 93, 94, 95, 96, 97, 98, or 99% identical to SEQ ID NO: 2 and/or SEQ ID NO: 3. In some embodiments, the primer set comprises a sequence of SEQ ID NO: 4 and SEQ ID NO: 5 or a sequence that is at least 90, 91, 92, 93, 94, 95, 96, 97, 98, or 99% identical to SEQ ID NO: 4 and/or SEQ ID NO: 5. In some embodiments, the primer set comprises a sequence of SEQ ID NO: 4 and SEQ ID NO: 43 or a sequence that is at least 90, 91, 92, 93, 94, 95, 96, 97, 98, or 99% identical to SEQ ID NO: 4 and/or SEQ ID NO: 43. In some embodiments, the primer set comprises a sequence of SEQ ID NO: 6 and SEQ ID NO: 7 or a sequence that is at least 90, 91, 92, 93, 94, 95, 96, 97, 98, or 99% identical to SEQ ID NO: 6 and/or SEQ ID NO: 7. In some embodiments, the primer set comprises a sequence of SEQ ID NO: 8 and SEQ ID NO: 9 or a sequence that is at least 90, 91, 92, 93, 94, 95, 96, 97, 98, or 99% identical to SEQ ID NO: 8 and/or SEQ ID NO: 9. Although the sequences provided for herein may be illustrated with a 5′ biotinylation, other tags can also be used that perform a similar function to the biotin or other modified forms of biotin can be used.

In some embodiments, the primer sets are used to produce framework accepter fragments that can be used to generate CDR sets. In some embodiments, 4 different human framework region acceptor fragments are produced. In some embodiments, the sequences of the acceptor fragments that are produced as provided for in Table 3 in the Examples section. In some embodiments, a framework acceptor fragment that can, in some embodiments correspond to the FR1-CDRH1-FR2 fragment, comprise SEQ ID NO: 10 or a sequence that is at least 90, 91, 92, 93, 94, 95, 96, 97, 98, or 99% identical to SEQ ID NO: 10. This framework acceptor fragment can, for example, comprise a human VH3-23 germline CDR1 region (CDRH1) in addition to flanking frameworks FR1 and FR2. This fragment can be produced, for example, using a primer set comprising SEQ ID NO: 2 and/or SEQ ID NO: 3 or a primer set that is at least 90, 91, 92, 93, 94, 95, 96, 97, 98, or 99% identical to SEQ ID NO: 2 and/or 3.

In some embodiments, a framework acceptor fragment that can correspond to framework 3 region (FR3) comprises a sequence of SEQ ID NO: 11 or SEQ ID NO: 44 or a sequence that is at least 90, 91, 92, 93, 94, 95, 96, 97, 98, or 99% identical to SEQ ID NO: 11 or SEQ ID NO: 44. This fragment can, for example, contains a single framework region, FR3, with, for example, an overhang at the terminal where the FR3 region can connect to the CDR2 region of the human VH3-23 gene. In some embodiments, this acceptor fragment is generated using a primer set comprising SEQ ID NO: 4 and SEQ ID NO: 5 or a primer set comprising SEQ ID NO: 4 and SEQ ID NO: 43 or a sequence that is at least 90, 91, 92, 93, 94, 95, 96, 97, 98, or 99% identical to SEQ ID NO: 4 and/or SEQ ID NO: 5 or SEQ ID NO: 4 and/or SEQ ID NO: 43. In some embodiments, an acceptor fragment is provided that comprises a portion of the FR3 region, which can for example contain a single framework region FR3 with an overhang at the terminal where the FR3 region connects to the CDR3 region of the human VH3-23 gene. This fragment, for example, can comprise a sequence of SEQ ID NO: 12 or a sequence that is at least 90, 91, 92, 93, 94, 95, 96, 97, 98, or 99% identical to SEQ ID NO: 12. In some embodiments, this fragment can be produced, for example, using a primer set comprising SEQ ID NO: 6 and/or SEQ ID NO: 7 or a primer set that is at least 90, 91, 92, 93, 94, 95, 96, 97, 98, or 99% identical to SEQ ID NO: 6 and/or 7.

In some embodiments, a fragment is provided for that corresponds to the FR4 portion of an antibody. In some embodiments the fragment comprises a sequence of SEQ ID NO: 13 or a sequence that is at least 90, 91, 92, 93, 94, 95, 96, 97, 98, or 99% identical to SEQ ID NO: 13. In some embodiments, this fragment can be produced, for example, using a primer set comprising SEQ ID NO: 8 and/or SEQ ID NO: 9 or a primer set that is at least 90, 91, 92, 93, 94, 95, 96, 97, 98, or 99% identical to SEQ ID NO: 8 and/or 9.

The CDR2 region of an antibody, in some embodiments can be produced from B cells of an immunized chicken. In some embodiments, a CDR2 region is amplified from cDNA that is prepared from RNA that is derived from (extracted from) the B cells of an immunized chicken. The CDR2 fragment, can for example be amplified using a primers, such as those listed in Table 4, which include, but are not limited to primers comprising a sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 31, SEQ ID NO: 32, SEQ ID NO: 33, SEQ ID NO: 34, SEQ ID NO: 35, or SEQ ID NO: 36 or a sequence that is at least at least 90, 91, 92, 93, 94, 95, 96, 97, 98, or 99% identical to of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 31, SEQ ID NO: 32, SEQ ID NO: 33, SEQ ID NO: 34, SEQ ID NO: 35, or SEQ ID NO: 36. In some embodiments, the primer set comprises a sequence of SEQ ID NO: 14, or a sequence that is at least 90, 91, 92, 93, 94, 95, 96, 97, 98, or 99% identical to of SEQ ID NO: 14 and one of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 31, SEQ ID NO: 32, SEQ ID NO: 33, SEQ ID NO: 34, SEQ ID NO: 35, or SEQ ID NO: 36, or a sequence that is at least 90, 91, 92, 93, 94, 95, 96, 97, 98, or 99% identical to one of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 31, SEQ ID NO: 32, SEQ ID NO: 33, SEQ ID NO: 34, SEQ ID NO: 35, or SEQ ID NO: 36. The sequence provide for degeneracy as certain positions, wherein the degenerate nucleic acid base is K, M, R, Y, S, W, B, D or H, wherein K is G or T/U; M is A or C; R is A or G; Y is C or T/U; S is C or G; W is A or T/U; B is C or G or T/U; D is A or G or T/U; A or C or T/U.

Without being bound to any particular theory, these pools of primers contain degenerate bases in order to sample as much of the chicken immune repertoire as possible. These primers, for example, can contain an AcuI restriction enzyme recognition site (CTGAAG in Table 4) positioned such that the cleavage of the restriction enzyme leaves an overhang at the desired junction point for ligation to the human framework region acceptor fragments. Other restrictions sites can be used to generate similar overhangs. These primers can also be, for example, biotinylated on the 5′ end for capture onto streptavidin magnetic beads and release by digestion with AcuI. A 5′ biotin is just one example and other binding moieties can be used in place of the biotin and be used with known capture systems. In some embodiments, a random spacer sequence between the biotin (capture binding moiety) and AcuI recognition site (or other used restriction site) is included to improve digestion efficiency.

In some embodiments, after the fragment is produced is digested with a restriction enzyme, such as, but not limited to AcuI, to produce the CDR2 with an overhang at the terminal where CDR2 connects to the FR3 region. The CDR3 fragment, can then, for example be ligated to the FR3 acceptor fragment, such as, but not limited to SEQ ID NO: 11 or SEQ ID NO: 44, or a sequence that is at least 90, 91, 92, 93, 94, 95, 96, 97, 98, or 99% identical to SEQ ID NO: 11 or SEQ ID NO: 44. This ligation product can then be amplified. The amplification product that is produced, can for example, be a CDR2-FR3, with an overhang at the terminal where the CDR2 connects to FR2.

In some embodiments, the CDR2-FR3 fragment is ligated to a FR1-CDR1-FR2 human framework region acceptor fragment, such as a sequence of SEQ ID NO: 10 or a sequence that is at least 90, 91, 92, 93, 94, 95, 96, 97, 98, or 99% identical to SEQ ID NO: 10. (SEQ ID NO: 10). After ligation, the ligated product, a FR1-CDR1-FR2-CDR2-FR3 fragment, can be used as a template for a PCR amplification to generate a VH CDR2 flanked by human VH3-23 FR1-CDR1-FR2 and FR3. This can be then be combined with a CDR3 region that is produced as provided for herein.

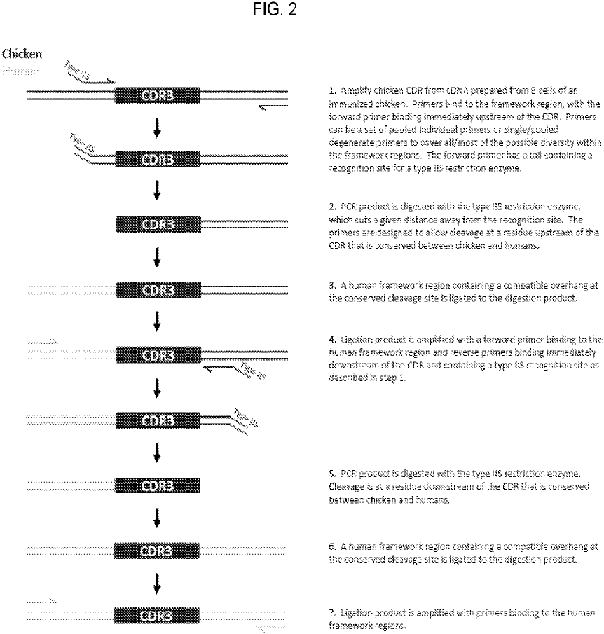

In some embodiments, a CDR3 region can be amplified from cDNA prepared from RNA extracted from B cells of an immunized chicken. In some embodiments, the CD3 extracted by PCR and digestion reactions. The CDR3 fragment can then be ligated onto the human framework region acceptor fragments, generating a fragment containing chicken CDR3 flanked by human framework regions. A non-limiting example of this is illustrated in FIG. 2. In some embodiments, the primers that can be used include, but not limited to, a sequence comprising SEQ ID NO: 23-30 or 37-42. In some embodiments, the primer set comprises one of SEQ ID NO: 28 or SEQ ID NO: 42 and one of SEQ ID NO: 23, SEQ ID NO: 24, SEQ ID NO: 25, SEQ ID NO: 26, SEQ ID NO: 27, SEQ ID NO: 29, SEQ ID NO: 30, SEQ ID NO: 37, SEQ ID NO: 38, SEQ ID NO: 39, SEQ ID NO: 40, SEQ ID NO: 41, or SEQ ID NO: 42. In some embodiments, the primer set comprises a sequence that is at least 90, 91, 92, 93, 94, 95, 96, 97, 98, or 99% identical to one of SEQ ID NO: 28 or SEQ ID NO: 42 and a sequence that is at least 90, 91, 92, 93, 94, 95, 96, 97, 98, or 99% identical to one of SEQ ID NO: 23, SEQ ID NO: 24, SEQ ID NO: 25, SEQ ID NO: 26, SEQ ID NO: 27, SEQ ID NO: 29, SEQ ID NO: 30, SEQ ID NO: 37, SEQ ID NO: 38, SEQ ID NO: 39, SEQ ID NO: 40, SEQ ID NO: 41, or SEQ ID NO: 42. The sequences provide for contain degeneracy as certain positions, wherein the degenerate nucleic acid base is K, M, R, Y, S, W, B, D or H, wherein K is G or T/U; M is A or C; R is A or G; Y is C or T/U; S is C or G; W is A or T/U; B is C or G or T/U; D is A or G or T/U; A or C or T/U. As provided herein, the primers can contain a restriction enzyme recognition site, such as, but not limited to, an AcuI restriction enzyme recognition site, positioned such that the cleavage of the restriction enzyme leaves an overhang at the desired junction point for ligation to the human framework region acceptor fragments. These primers can be biotinylated on the 5′ end for capture onto streptavidin magnetic beads and release by digestion with AcuI. Other tags or capture reagents can be used to perform a similar function. In some embodiments, a random spacer sequence can be used between the biotin and AcuI recognition site to, for example, improve digestion efficiency.

In some embodiments, methods of identifying a binding partner to a target are provided. In some embodiments, the methods comprise contacting the target with a library of proteins encoded by a library prepared according to a method as provided herein and detecting a binding partner to the target. The target can be any target. In some embodiments, the target is a multiple membrane spanning protein. In some embodiments, the target protein is present on the surface of a virus-like particle, such as a lipoparticle. Lipoparticles comprising multiple membrane spanning proteins can be generated according to any method. For example, U.S. Pat. Nos. 9,902,765, 9,213,027, 8,574,590, and 8,377,691, each of which is hereby incorporated by reference in its entirety. In some embodiments, the binding partner is an antibody. In some embodiments, the antibody is an scFv format or other formats as provided for herein.

As described herein, the libraries provided herein can be used to produce and/or identify antibodies. The antibodies can be used for various purposes, such as therapeutics, diagnostics reagents, detection reagents, and the like.

The term “antibody” as used herein is meant in a broad sense and includes immunoglobulin or antibody molecules including polyclonal antibodies, monoclonal antibodies including murine, human, humanized and chimeric monoclonal antibodies and antibody fragments, such as ScFv or hexabodies (PLOS Biology | DOI:10.1371/journal.pbio.1002344 Jan. 6, 2016, which is hereby incorporated by reference in its entirety).

“Encoding” refers to the inherent property of specific sequences of nucleotides in a polynucleotide, such as a gene, a cDNA, an mRNA, or viral RNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments, the expression vector is the alphaviruses as described herein.

The term “humanized antibody”, “engineered antibody”, “human framework adapted”, and “HFA” as used herein, is intended to include antibodies having variable region frameworks derived from sequences of human origin. Furthermore, if the antibody contains a constant region, the constant region can be derived from such human sequences, e.g., human germline sequences, or naturally occurring (e.g., allotypes) or mutated versions of human germline sequences. The humanized antibodies may include amino acid residues not encoded by human sequences (e.g., mutations introduced by random or site-specific mutagenesis in vitro or by somatic mutation in vivo).

In general, antibodies are proteins or polypeptides that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

The term “antibody fragment” means a portion of an intact antibody, generally the antigen binding or variable region of the intact antibody. Examples of antibody fragments include Fab, Fab′, F(ab′)2 and Fv fragments, diabodies, single chain antibody molecules and multispecific antibodies formed from at least two intact antibodies.

The term “antigen” as used herein means any molecule that has the ability to generate antibodies either directly or indirectly. Included within the definition of “antigen” is a protein-encoding nucleic acid.

As used herein, “specific binding” or “immunospecific binding” or “binds immunospecifically” refer to antibody binding to a predetermined antigen or epitope present on the antigen. In some embodiments, the antibody binds with a dissociation constant (KD) of 10-7 M or less, and binds to the predetermined antigen with a KD that is at least two-fold less than its KD for binding to a non-specific antigen (e.g., BSA, casein, or another non-specific polypeptide) other than the predetermined antigen. The phrases “an antibody recognizing an antigen/protein/target” and “an antibody specific for antigen/protein/target” are used interchangeably herein with the term “an antibody which binds immunospecifically to antigen/protein/target.”

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived.

The term “homolog” means protein sequences having between 40% and 100% sequence identity to a reference sequence. Percent identity between two peptide chains can be determined by pair wise alignment using the default settings of the AlignX module of Vector NTI v.9.0.0 (Invitrogen Corp., Carslbad, Calif.). In some embodiments, the an antibody or fragment thereof has at least 50, 60, 70, 80, 90, 91, 92, 93, 94, 95, 96, 97, 98, or 99% identity to a sequence described herein. In some embodiments, the antibody has conservative substitutions as compared to a sequence described herein. In some embodiments, the number of substitutions can be 1, 2, 3, 4, 5, 6, 7, 8, or 9. These molecules that differ based on % identity or substitutions can also be referred to as “variants.” Antibodies having conservative substitutions in the heavy and light chain sequences shown in Table 1 are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

Table: Exemplary Conservative Substitutions:

| | |
|---|---|
| Original Residue | Exemplary Conservative Substitutions |
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

The term “in combination with” as used herein means that the described agents can be administered to an animal together in a mixture, concurrently as single agents or sequentially as single agents in any order.

Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. MAbs may be obtained by methods known to those skilled in the art. See, for example Kohler and Milstein, Nature 256:495 497 (1975); U.S. Pat. No. 4,376,110; Ausubel et al., eds., Current Protocols in Molecular Biology, Greene Publishing Assoc. and Wiley Interscience, N.Y., (1987, 1992); and Harlow and Lane ANTIBODIES: A Laboratory Manual Cold Spring Harbor Laboratory (1988); Colligan et al., eds., Current Protocols in Immunology, Greene Publishing Assoc. and Wiley Interscience, N.Y., (1992, 1993), the contents of which references are incorporated entirely herein by reference. Such antibodies may be of any immunoglobulin class including IgG, IgM, IgE, IgA, GILD and any subclass thereof. A hybridoma producing a mAb may be cultivated in vitro, in situ or in vivo. Production of high titers of mAbs in vivo or in situ makes this the presently preferred method of production.

Chimeric antibodies are molecules different portions of which are derived from different animal species, such as those having variable region derived from a murine mAb and a human immunoglobulin constant region, which are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Cabilly et al., Proc. Natl. Acad. Sci. USA 81:3273 3277 (1984); Morrison et al., Proc. Natl. Acad. Sci. USA 81:6851 6855 (1984); Boulianne et al., Nature 312:643 646 (1984); Cabilly et al., European Patent Application 125023 (published Nov. 14, 1984); Neuberger et al., Nature 314:268 270 (1985); Taniguchi et al., European Patent Application 171496 (published Feb. 19, 1985); Morrison et al., European Patent Application 173494 (published Mar. 5, 1986); Neuberger et al., PCT Application WO 86/01533, (published Mar. 13, 1986); Kudo et al., European Patent Application 184187 (published Jun. 11, 1986); Morrison et al., European Patent Application 173494 (published Mar. 5, 1986); Sahagan et al., J. Immunol. 137:1066 1074 (1986); Robinson et al., International Patent Publication WO 1987/002671 (published May 7, 1987); Liu et al., Proc. Natl. Acad. Sci. USA 84:3439 3443 (1987); Sun et al., Proc. Natl. Acad. Sci. USA 84:214 218(1987); Better et al., Science 240:1041 1043 (1988); and Harlow and Lane Antibodies. a Laboratory Manual Cold Spring Harbor Laboratory (1988)). These references are entirely incorporated herein by reference.

An anti-idiotypic (anti-Id) antibody is an antibody which recognizes unique determinants generally associated with the antigen-binding site of an antibody. An Id antibody can be prepared by immunizing an animal of the same species and genetic type (e.g., mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference. The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity.

The term “monoclonal antibody” (mAb) as used herein means an antibody (or antibody fragment) obtained from a population of substantially homogeneous antibodies. Monoclonal antibodies are highly specific, typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256:495-497 (1975). Chimeric mAbs containing a light chain and heavy chain variable region derived from a donor antibody (typically murine) in association with light and heavy chain constant regions derived from an acceptor antibody (typically another mammalian species such as human) can be prepared by the method disclosed in U.S. Pat. No. 4,816,567. Humanized mAbs having CDRs derived from a non-human donor immunoglobulin (typically murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl. Acad. Sci. (USA), 86:10029-10032 (1989) and Hodgson et al., Bio/Technology, 9:421 (1991).

In addition to the antibodies described herein, exemplary human framework sequences useful for humanization are disclosed at, e.g., www″dot″ncbi″dot″nlm″dot″nih″dot″gov/entrez/query″dot″fcgi; www″dot″ncbi″dot″nih″dot″gov/igblast; www″dot″atec″dot″org/phage/hdb″dot″html; www″dot″mrc-cpe″dot″cam″dot″ac″dot″uk/ALIGNMENTS″dot″php; “dot” www″dot″kabatdatabase″dot″com/top″dot″html; ftp″dot″ncbi″dot″nih″dot″gov/repository/kabat; www″dot″sciquest″dot″com; www″dot″abcam″dot″com; www″dot″antibodyresource″dot″com/onlinecomp″dot″html; www″dot″public″dot″iastate″dot″edu/″dot″about″dot″pedro/research_tools″dot″html; www″dot″whfreeman″dot″com/immunology/CH05/kuby05″dot″htm; www″dot″hhmi″dot″org/grants/lectures/1996/vlab; www″dot″path″dot″cam″dot″ac″dot″uk/″dot″about″dot″mrc7/mikeimages″dot″html; mcb″dot″harvard″dot″edu/BioLinks/Immunology″dot″html; www″dot″immunologylink″dot″com; pathbox″dot″wustl″dot″edu/″dot″about″dot″hcenter/index″dot″html; www″dot″appliedbiosystems″dot″com; www″dot″nal″dot″usda″dot″gov/awic/pubs/antibody; www″dot″m″dot″ehime-u″dot″ac″dot″jp/″dot″about″dot″yasuhito/Elisa″dot″html; www″dot″biodesign″dot″com; www″dot″cancerresearchuk″dot″org; www″dot″biotech″dot″ufl″dot″edu; www″dot″isac-net″dot″org; baserv″dot″uci″dot″kun″dot″nl/″dot″about″dot″jraats/links1″dot″html; www″dot″recab″dot″uni-hd″dot″de/immuno″dot″bme″dot″nwu″dot″edu; www″dot″mrc-cpe″dot″cam″dot″ac″dot″uk; www″dot″ibt″dot″unam″dot″mx/vir/V_mice″dot″html; http://www″dot″bioinf″dot″org″dot″uk/abs; antibody″dot″bath″dot″ac″dot″uk; www″dot″unizh″dot″ch; www″dot″cryst″dot″bbk″dot″ac″dot″uk/″dot″about″dot″ubcg07s; www″dot″nimr″dot″mrc″dot″ac″dot″uk/CC/ccaewg/ccaewg″dot″html; www″dot″path″dot″cam″dot″ac″dot″uk/″dot″about″dot″mrc7/humanisation/TAHHP″dot″html; www″dot″ibt″dot″unam″dot″mx/vir/structure/stat_aim″dot″html; www″dot″biosci″dot″missouri″dot″edu/smithgp/index″dot″html; www″dot″jerini″dot″de; imgt″dot″cines″dot″fr; and Kabat et al., Sequences of Proteins of Immunological Interest, U.S. Dept. Health (1987), each entirely incorporated herein by reference. The “dot” in the world wide web addresses referenced herein can be replaced with a “.” as appropriate.

The antibodies described herein can include, but are not limited to, at least one of a heavy chain constant region (H_{c}), a heavy chain variable region (Hᵥ), a light chain variable region (Lᵥ) and a light chain constant region (L_{c}), wherein a polyclonal Ab, monoclonal Ab, fragment and/or regions thereof include at least one heavy chain variable region (Hᵥ) or light chain variable region (Lᵥ) which binds a portion of a target and can be used to detect the antigen. The antibodies can also be monoclonal antibodies that are made by immunizing chickens. The variable chains from the nucleic acid sequences encoding the isolated monoclonal antibodies can be isolated by using techniques, such as but not limited to, PCR. The variable chains isolated by these techniques can then be placed in a scFv vector with a human Fc. Accordingly, the antibodies can be antibodies that have a human Fc and two scFv arms. The antibodies, such as those described here and throughout the present disclosure can then be modified to be human or humanized antibodies. Examples of how to modify an antibody, including chicken antibodies, can be found in, for example, Riechmann L, Clark M, Waldmann H, Winter G (1988). Reshaping human antibodies for therapy″. Nature 332 (6162): 332-323; Tsurushita N, Park M, Pakabunto K, Ong K, Avdalovic A, Fu H, Jia A, Vásquez M, Kumar S. (2004); and “Humanization of a chicken anti-IL-12 monoclonal antibody” Immunol Methods 295 (1-2): 9-19; Nishibori N, Horiuchi H, Furusawa S, Matsuda H. (2006) “Humanization of chicken monoclonal antibody using phage display system” Mol Immunol. 43 (6): 634-42, each of which is incorporated by reference in its entirety.

Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589 601 (1983), which references are entirely incorporated herein by reference.

As used herein, the term “antigen binding region” refers to that portion of an antibody molecule which contains the amino acid residues that interact with an antigen and confer on the antibody its specificity and affinity for the antigen. The antibody region includes the “framework” amino acid residues necessary to maintain the proper conformation of the antigen-binding residues. In some embodiments, the antigen binding region will be of murine origin. In some embodiments, the antigen binding region can be derived from other animal species, in particular rodents such as rabbit, rat or hamster, or birds such as chickens. It has been shown that the antigen-binding function of an antibody can be performed by fragments of a full-length antibody. Examples of binding fragments encompassed within the term “antigen-binding portion” of an antibody include a Fab fragment, a monovalent fragment having the VL, VH, CL and CH1 domains; a F(ab)₂ fragment, a bivalent fragment comprising two Fab fragments linked by a disulfide bridge(s) at a hinge region; a Fd fragment having the VH and CH1 domains; a Fv fragment having the VL and VH domains of a single arm of an antibody; a domain antibody or dAb fragment (Ward et al., 1989 Nature 341:544-546), which consists of a VH domain; and an isolated complementarity determining region (CDR), especially a CDR3 (See for example the WO03/025019, the contents of which are incorporated herein by reference).

“Framework Region” or “FR” residues are those variable domain residues other than the hypervariable region residues as herein defined. The sequences of the framework regions of different light (i.e. L-FR1, L-FR2, L-FR3 and L-FR4) or heavy (i.e. H-FR1, H-FR2, H-FR3 and H-FR4) chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The four framework sub-regions (FR1, FR2, FR3, and FR4) of the variable region of both the heavy and light chain are interrupted by three stretches of hypervariable sequences, or the complementary determining regions (CDR’s), as defined in Kabat’s database (Kabat et al., Variable region genes for the immunoglobulin framework are assembled from small segments of DNA-a hypothesis, PNAS 75(5):2429-33, 1978), with the CDR1 positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR’s within the variable region of a single, naturally occurring immunoglobulin chain. As used herein, a FR represents one of the four sub-regions, and FR’s represents two or more of the four sub-regions constituting a framework region. The sequences of the framework regions of different light or heavy chains are relatively conserved within a species.

The term “Complementarity Determining Regions (CDRs)” is based on sequence variability (Wu and Kabat, J. Exp. Med. 132:211-250, 1970). There are six CDRs--three in the variable heavy chain, or VH, and are typically designated H-CDR1, H-CDR2, and H-CDR3, and three CDRs in the variable light chain, or VL, and are typically designated L-CDR1, L-CDR2, and L-CDR3 (Kabat et al., Sequences of Proteins of Immunological Interest, 5th Ed. Public Health Service, National Institutes of Health, Bethesda, Md., 1991). “Hypervariable region”, “HVR”, or “HV” refer to the regions of an antibody variable domain which are variable in structure as defined by Chothia and Lesk (Chothia and Lesk, Mol. Biol. 196:901-917, 1987). There are six HVRs, three in VH (H1, H2, H3) and three in VL (L1, L2, L3). Chothia and Lesk refer to structurally conserved HVs as “canonical structures.” Another method of describing the regions that form the antigen-binding site has been proposed by Lefranc (Lefranc et al., Developmental & Comparative Immunology 27:55-77, 2003) based on the comparison of V domains from immunoglobulins and T-cell receptors (Lefranc et al., Developmental & Comparative Immunology 27:55-77, 2003). The antigen-binding site can also be delineated based on “Specificity Determining Residue Usage (SDRU)”, according to Almagro (Almagro, Mol. Recognit. 17:132-43, 2004), where SDRU refers to amino acid residues of an immunoglobulin that are directly involved in antigen contact.

Furthermore, although the two domains of the Fv fragment, VL and VH, are encoded by separate genes naturally, they can be joined, using recombinant methods, by a synthetic linker that enables them to be made as a single protein chain in which the VL and VH regions pair to form monovalent molecules (known as single chain Fv (scFv); see e.g., Bird et al., 1988 Science 242:423-426; and Huston et al., 1988 Proc. Nat. Acad. Sci. 85:5879-5883). Such single chain antibodies are encompassed by the term “antigen-binding portion” of an antibody. These antibody fragments are obtained using conventional techniques known to those of skill in the art, and can be used in the same manner as intact antibodies.

An “isolated antibody,” as used herein, refers to an antibody that is substantially free of other antibodies having different antigenic specificities. Moreover, an isolated antibody may be substantially free of other cellular material and/or chemicals. An isolated antibody can also be sterile or pyrogen free or formulated as injectable pharmaceutical as described herein.

An “antigen” is a molecule or a portion of a molecule capable of being bound by an antibody which is additionally capable of inducing an animal to produce antibody capable of binding to an epitope of that antigen. An antigen can have one or more than one epitope. The specific reaction referred to above is meant to indicate that the antigen will react, in a highly selective manner, with its corresponding antibody and not with the multitude of other antibodies which can be evoked by other antigens. In some embodiments, antigens that bind antibodies, fragments and regions of the antibodies include at least 5 amino acids. In some embodiments, the cell is an intact cell. An intact cell is a cell that has not been lysed or broken open with the use of detergents or other reagents. A cell that has been treated with detergents or other reagents that breaks up the cellular membrane or punches holes in a cellular membrane is not an intact cell. By expressing the receptor on the surface of the cell or particle, e.g. lipoparticle, the receptor can present conformational epitopes that may otherwise not be present if purified protein is used. An example is provided herein. In some embodiments, an adjuvant is not used, but an adjuvant can be used. In some embodiments, the particles are injected into a bird (e.g. chicken) to stimulate an immune response and generate antibodies against the protein present on the surface of the particle. Particles suitable for the generation of antibodies are described in U.S. Pat. Nos.: 8,377,691, 7,763,258, 8,158,130 and U.S. Pat. Application Publication Nos. 20050123563 and 20120195882, each of which is hereby incorporated by reference. These publications and patents describe the generation of various particles, including lipoparticles, that can be used to express membrane spanning proteins (e.g. multiple-membrane spanning proteins, ion channels, and the like).

The term “epitope” is meant to refer to that portion of any molecule capable of being recognized by and bound by an antibody at one or more of the Ab’s antigen binding regions. Epitopes usually consist of chemically active surface groupings of molecules such as amino acids or sugar side chains and have specific three dimensional structural characteristics as well as specific charge characteristics. Example of epitopes include, but are not limited to,

As used herein, the term “chimeric antibody” includes monovalent, divalent or polyvalent immunoglobulins. A monovalent chimeric antibody is a dimer (HL) formed by a chimeric H chain associated through disulfide bridges with a chimeric L chain. A divalent chimeric antibody is tetramer (H₂L₂) formed by two HL dimers associated through at least one disulfide bridge. A polyvalent chimeric antibody can also be produced, for example, by employing a C_{H} region that aggregates (e.g., from an IgM H chain, or µ. chain). In some embodiments, murine and chimeric antibodies, fragments and regions comprise individual heavy (H) and/or light (L) immunoglobulin chains.

Antibodies, fragments or derivatives having chimeric H chains and L chains of the same or different variable region binding specificity, can also be prepared by appropriate association of the individual polypeptide chains, according to known method steps, e.g., according to Ausubel infra, Harlow infra, and Colligan infra, the contents of which references are incorporated entirely herein by reference. With this approach, hosts expressing chimeric H chains (or their derivatives) are separately cultured from hosts expressing chimeric L chains (or their derivatives), and the immunoglobulin chains are separately recovered and then associated. Alternatively, the hosts can be co-cultured and the chains allowed to associate spontaneously in the culture medium, followed by recovery of the assembled immunoglobulin, fragment or derivative.

The hybrid cells are formed by the fusion of a non-human antibody-producing cell, typically a spleen cell of an animal immunized against either natural or recombinant antigen, or a peptide fragment of the antigen protein sequence. Alternatively, the non-human antibody-producing cell can be a B lymphocyte obtained from the blood, spleen, lymph nodes or other tissue of an animal immunized with the antigen.

The second fusion partner, which provides the immortalizing function, can be a lymphoblastoid cell or a plasmacytoma or myeloma cell, which is not itself an antibody producing cell, but is malignant. Fusion partner cells include, but are not limited to, the hybridoma SP2/0-Ag14, abbreviated as SP2/0 (ATCC CRL1581) and the myeloma P3X63Ag8 (ATCC TIB9), or its derivatives. See, e.g., Ausubel infra, Harlow infra, and Colligan infra, the contents of which references are incorporated entirely herein by reference.

The antibodies can be generated according the examples provided herein. Once the sequences are known, the antibodies can also be generated according to known methods. The antibodies can also be converted to different types, such as being converted to Human IgGs and the like. By converting the antibodies to a human antibody, a human subject should not identify the antibodies as foreign. This will lead to a more effective response. The conversion of a non-human IgG antibody to a human IgG antibody is well known and can routinely be done once the native sequence is known. As discussed herein, the antibodies can be modified according to known methods. Such methods are described in, for example, Riechmann L, Clark M, Waldmann H, Winter G (1988). Reshaping human antibodies for therapy″. Nature 332 (6162): 332-323; Tsurushita N, Park M, Pakabunto K, Ong K, Avdalovic A, Fu H, Jia A, Vásquez M, Kumar S. (2004); and “Humanization of a chicken anti-IL-12 monoclonal antibody” Immunol Methods 295 (1-2): 9-19; Nishibori N, Horiuchi H, Furusawa S, Matsuda H. (2006) “Humanization of chicken monoclonal antibody using phage display system” Mol Immunol. 43 (6): 634-42, each of which is incorporated by reference in its entirety.

The antibody-producing cell contributing the nucleotide sequences encoding the antigen-binding region of the chimeric antibody can also be produced by transformation of a non-human, such as a primate, or a human cell. For example, a B lymphocyte which produces the antibody can be infected and transformed with a virus such as Epstein-Barr virus to yield an immortal antibody producing cell (Kozbor et al., Immunol. Today 4:72 79 (1983)). Alternatively, the B lymphocyte can be transformed by providing a transforming gene or transforming gene product, as is well-known in the art. See, e.g., Ausubel infra, Harlow infra, and Colligan infra, the contents of which references are incorporated entirely herein by reference.

The cell fusions are accomplished by standard procedures well known to those skilled in the field of immunology. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

The antigen-specific murine or chimeric mAb can be produced in large quantities by injecting hybridoma or transfectoma cells secreting the antibody into the peritoneal cavity of mice and, after appropriate time, harvesting the ascites fluid which contains a high titer of the mAb, and isolating the mAb therefrom. For such in vivo production of the mAb with a non-murine hybridoma (e.g., rat or human), hybridoma cells are preferably grown in irradiated or athymic nude mice. Alternatively, the antibodies can be produced by culturing hybridoma or transfectoma cells in vitro and isolating secreted mAb from the cell culture medium or recombinantly, in eukaryotic or prokaryotic cells.

The sequences of the antibodies can be modified to yield human IgG antibodies. The conversion of the sequences provided herein can be modified to yield other types of antibodies. The CDRs can also be linked to other antibodies, proteins, or molecules to create antibody fragments that bind to the target. The CDRs and antibody sequences provided herein also be humanized or made fully human according to known methods. The sequences can also be made into chimeric antibodies as described herein.

In some embodiments, the antibody comprises an amino acid sequence comprising a sequence provided for herein or a fragment thereof. In some embodiments, the antibody comprises one or more amino acid sequences as provided herein, an antigen binding fragments, thereof, or a human IgG variant thereof. “A human IgG variant thereof” refers to an antibody that has been modified to be a human IgG when the starting antibody is not a human IgG antibody.

As described herein the production of antibodies with a known sequence is routine and can be done by any method. Accordingly, in some embodiments, a nucleic acid encoding an antibody or fragment thereof is provided. In some embodiments, the nucleic acid encodes a sequence provided for herein. The antibodies can also be modified to be chimeric antibodies or human antibodies. The antibodies can also be used in injectable pharmaceutical compositions. As also described herein, the antibodies can be isolated antibodies or engineered antibodies.

. In some embodiments, “derivatives” of the antibodies, fragments, regions or derivatives thereof, which term includes those proteins encoded by truncated or modified genes to yield molecular species functionally resembling the immunoglobulin fragments are provided. The modifications include, but are not limited to, addition of genetic sequences coding for cytotoxic proteins such as plant and bacterial toxins. The modification can also include a reporter protein, such as a fluorescent or chemiluminescent tag. The fragments and derivatives can be produced in any manner.

Fragments include, for example, Fab, Fab′, F(ab′)₂ and Fv. These fragments lack the Fc fragment of intact antibody, clear more rapidly from the circulation, and can have less non-specific tissue binding than an intact antibody (Wahl et al., J. Nucl. Med. 24:316 325 (1983)). These fragments are produced from intact antibodies using methods well known in the art, for example by proteolytic cleavage with enzymes such as papain (to produce Fab fragments) or pepsin (to produce F(ab′)_{f} fragments).

The identification of these antigen binding region and/or epitopes recognized by Abs described herein provide the information necessary to generate additional monoclonal antibodies with similar binding characteristics and therapeutic or diagnostic utility that parallel the embodiments of this application.

The nucleic acid sequence encoding an antibody described herein can be genomic DNA or cDNA, or RNA (e.g. mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize a target can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe, et al., J. Molec. Biol. 183:1 12 (1985). Using the “codon usage rules” of Lathe, a single oligonucleotide, or a set of oligonucleotides, that contains a theoretical “most probable” nucleotide sequence capable of encoding an antibody variable or constant region sequences is identified.

The variable regions described herein can be combined with any type of constant region including a human constant region or murine constant region. Human genes which encode the constant (C) regions of the antibodies, fragments and regions can be derived from a human fetal liver library, by known methods. Human C regions genes can be derived from any human cell including those which express and produce human immunoglobulins. The human C_{H} region can be derived from any of the known classes or isotypes of human H chains, including gamma, µ, α, δ or ε, and subtypes thereof, such as G1, G2, G3 and G4. Since the H chain isotype is responsible for the various effector functions of an antibody, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (IgG3), gamma 4 (IgG4), or µ (IgM). The human C_{L} region can be derived from either human L chain isotype, kappa or lambda.

Genes encoding human immunoglobulin C regions can be obtained from human cells by standard cloning techniques (Sambrook, et al. (Molecular Cloning: A Laboratory Manual, 2nd Edition, Cold Spring Harbor Press, Cold Spring Harbor, N.Y. (1989) and Ausubel et al., eds. Current Protocols in Molecular Biology (1987 1993)). Human C region genes are readily available from known clones containing genes representing the two classes of L chains, the five classes of H chains and subclasses thereof. Chimeric antibody fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the CH₁ domain and hinge region of the H chain, followed by a translational stop codon to yield the truncated molecule.

Generally, the murine, human or murine and chimeric antibodies, fragments and regions of the antibodies described herein are produced by cloning DNA segments encoding the H and L chain antigen-binding regions of an antigen specific antibody, and joining these DNA segments to DNA segments encoding C_{H} and C_{L} regions, respectively, to produce murine, human or chimeric immunoglobulin-encoding genes.

Thus, in some embodiments, a fused chimeric gene is created which comprises a first DNA segment that encodes at least the antigen-binding region of non-human origin, such as a functionally rearranged V region with joining (J) segment, linked to a second DNA segment encoding at least a part of a human C region.

Therefore, cDNA encoding the antibody V and C regions, the method of producing the chimeric antibody according to some of the embodiments described herein involve several steps, as exemplified below: 1. isolation of messenger RNA (mRNA) from the cell line producing an anti-antigen antibody and from optional additional antibodies supplying heavy and light constant regions; cloning and cDNA production therefrom; 2. preparation of a full length cDNA library from purified mRNA from which the appropriate V and/or C region gene segments of the L and H chain genes can be: (i) identified with appropriate probes, (ii) sequenced, and (iii) made compatible with a C or V gene segment from another antibody for a chimeric antibody; 3. Construction of complete H or L chain coding sequences by linkage of the cloned specific V region gene segments to cloned C region gene, as described above; 4. Expression and production of L and H chains in selected hosts, including prokaryotic and eukaryotic cells to provide murine-murine, human-murine, human-human or human murine antibodies.

One common feature of all immunoglobulin H and L chain genes and their encoded mRNAs is the J region. H and L chain J regions have different sequences, but a high degree of sequence homology exists (greater than 80%) among each group, especially near the C region. This homology is exploited in this method and consensus sequences of H and L chain J regions can be used to design oligonucleotides for use as primers for introducing useful restriction sites into the J region for subsequent linkage of V region segments to human C region segments.

C region cDNA vectors prepared from human cells can be modified by site-directed mutagenesis to place a restriction site at the analogous position in the human sequence. For example, one can clone the complete human kappa chain C (Cₖ) region and the complete human gamma-1 C region (Cγ-1). In this case, the alternative method based upon genomic C region clones as the source for C region vectors would not allow these genes to be expressed in bacterial systems where enzymes needed to remove intervening sequences are absent. Cloned V region segments are excised and ligated to L or H chain C region vectors. Alternatively, the human Cγ-1 region can be modified by introducing a termination codon thereby generating a gene sequence which encodes the H chain portion of an Fab molecule. The coding sequences with linked V and C regions are then transferred into appropriate expression vehicles for expression in appropriate hosts, prokaryotic or eukaryotic.

Two coding DNA sequences are said to be “operably linked” if the linkage results in a continuously translatable sequence without alteration or interruption of the triplet reading frame. A DNA coding sequence is operably linked to a gene expression element if the linkage results in the proper function of that gene expression element to result in expression of the coding sequence.

Expression vehicles include plasmids or other vectors. Preferred among these are vehicles carrying a functionally complete human C_{H} or C_{L} chain sequence having appropriate restriction sites engineered so that any V_{H} or V_{L} chain sequence with appropriate cohesive ends can be easily inserted therein. Human C_{H} or C_{L} chain sequence-containing vehicles thus serve as intermediates for the expression of any desired complete H or L chain in any appropriate host.

A chimeric antibody, such as a mouse-human or human-human, will typically be synthesized from genes driven by the chromosomal gene promoters native to the mouse H and L chain V regions used in the constructs; splicing usually occurs between the splice donor site in the mouse J region and the splice acceptor site preceding the human C region and also at the splice regions that occur within the human C region; polyadenylation and transcription termination occur at native chromosomal sites downstream of the human coding regions.

As used herein and unless otherwise indicated, the term “about” is intended to mean ± 5% of the value it modifies. Thus, about 100 means 95 to 105.

In some embodiments, the antibodies described herein are used to detect the presence of the antigen. The present antibody can be used in any device or method to detect the presence of the antigen.

The term “purified” with referenced to an antibody refers to an antibody that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and, at least 95%, 96%, 97%, 98%, 99%, or 100% (w/w) pure. In some embodiments, the antibody is purified.

The terms “specific binding,” “specifically binds,” and the like, mean that two or more molecules form a complex that is measurable under physiologic or assay conditions and is selective. An antibody or antigen binding protein or other molecule is said to “specifically bind” to a protein, antigen, or epitope if, under appropriately selected conditions, such binding is not substantially inhibited, while at the same time non-specific binding is inhibited. Specific binding is characterized by a high affinity and is selective for the compound, protein, epitope, or antigen. Nonspecific binding usually has a low affinity. Binding in IgG antibodies for example is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody or antigen binding protein carrying the antigen-binding domain will generally not bind other antigens. In some embodiments, the capture reagent has a Kd equal or less than 10⁻⁹M, 10⁻¹⁰M, or 10⁻¹¹M for its binding partner (e.g. antigen). In some embodiments, the capture reagent has a Ka greater than or equal to 10⁹M⁻¹ for its binding partner.

Intact antibodies, also known as immunoglobulins, are typically tetrameric glycosylated proteins composed of two light (L) chains of approximately 25 kDa each, and two heavy (H) chains of approximately 50 kDa each. Two types of light chain, termed lambda and kappa, exist in antibodies. Depending on the amino acid sequence of the constant domain of heavy chains, immunoglobulins are assigned to five major classes: A, D, E, G, and M, and several of these may be further divided into subclasses (isotypes), e.g., IgG1, IgG2, IgG3, IgG4, IgA1, and IgA2. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated CH1. The VH and VL domains consist of four regions of relatively conserved sequences named framework regions (FR1, FR2, FR3, and FR4), which form a scaffold for three regions of hypervariable sequences (complementarity determining regions, CDRs). The CDRs contain most of the residues responsible for specific interactions of the antibody or antigen binding protein with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody or antigen binding protein-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, Eds. Harlow et al., 1988. One of skill in the art will recognize that each subunit structure, e.g., a CH, VH, CL, VL, CDR, and/or FR structure, comprises active fragments. For example, active fragments may consist of the portion of the VH, VL, or CDR subunit that binds the antigen, i.e., the antigen-binding fragment, or the portion of the CH subunit that binds to and/or activates an Fc receptor and/or complement.

In addition to the fragments described herein, non-limiting examples of binding fragments encompassed within the term “antigen-specific antibody” used herein include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). The most commonly used linker is a 15-residue (Gly4Ser)₃ peptide, but other linkers are also known in the art. Single chain antibodies are also intended to be encompassed within the terms “antibody or antigen binding protein,” or “antigen-binding fragment” of an antibody. The antibody can also be a polyclonal antibody, monoclonal antibody, chimeric antibody, antigen-binding fragment, Fc fragment, single chain antibodies, or any derivatives thereof.

These antibodies can be obtained using conventional techniques known to those skilled in the art and described herein, and the fragments are used in the same manner as intact antibodies. Antibody diversity is created by multiple germline genes encoding variable domains and a variety of somatic events. The somatic events include recombination of variable gene segments with diversity (D) and joining (J) gene segments to make a complete VH domain, and the recombination of variable and joining gene segments to make a complete VL domain. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation. Based on the estimated number of germline gene segments, the random recombination of these segments, and random VH-VL pairing, up to 1.6×10⁷ different antibodies may be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×10¹⁰ different antibodies may be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

Antibody or antigen binding protein molecules capable of specifically interacting with the antigens, epitopes, or other molecules described herein may be produced by methods well known to those skilled in the art. For example, monoclonal antibodies can be produced by generation of hybridomas in accordance with known methods. Hybridomas formed in this manner can then be screened using standard methods, such as enzyme-linked immunosorbent assay (ELISA) and biosensor analysis, to identify one or more hybridomas that produce an antibody that specifically interacts with a molecule or compound of interest.

As an alternative to preparing monoclonal antibody-secreting hybridomas, a monoclonal antibody to a polypeptide may be identified and isolated by screening a recombinant combinatorial immunoglobulin library (e.g., an antibody phage display library) with a polypeptide described herein to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature. Thus, the epitopes described herein can be used to screen for other antibodies that can be used therapeutically, diagnostically, or as research tools.

### Administration, Compositions, and Kits Comprising the Antibodies

Whereas, an isolated antibody binds an epitope on target, the antibodies or antigen binding fragments thereof, produced according to the embodiments provided herein, can be suitable both as therapeutic and prophylactic agents for treating conditions in humans and animals.

In some embodiments, the methods comprise administering a therapeutically or prophylactically effective amount of one or more antibodies or antigen binding fragments of the antibodies provided for herein to a susceptible subject or to one exhibiting a condition in which the antibody is thought to be useful. Any active form of the antibody can be administered, including, but not limited to Fab and F(ab′)2 fragments.

In some embodiments, the antibodies used are compatible with the recipient species such that the immune response to the MAbs does not result in an unacceptably short circulating half-life or induce an immune response to the MAbs in the subject. In some embodiments, the MAbs administered exhibit some secondary functions such as binding to Fc receptors of the subject and activation of antibody dependent cell mediated cytotoxicity (ADCC) mechanisms.

Treatment of individuals may comprise the administration of a therapeutically effective amount of the antibodies provided herein. The antibodies can be provided in a kit as described below. The antibodies can be used or administered alone or in admixture with another therapeutic, analgesic, or diagnostic agent.

Suitable vehicles and their formulation and packaging are described, for example, in Remington: The Science and Practice of Pharmacy (21st ed., Troy, D. ed., Lippincott Williams & Wilkins, Baltimore, Md. (2005) Chapters 40 and 41). Additional pharmaceutical methods may be employed to control the duration of action. Controlled release preparations may be achieved through the use of polymers to complex or absorb the compounds. Another possible method to control the duration of action by controlled release preparations is to incorporate the compounds of into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions.

In general, if administering a systemic dose of the antibody, it is desirable to provide the recipient with a dosage of antibody which is in the range of from about 1 ng/kg-100 ng/kg, 100 ng/kg-500 ng/kg, 500 ng/kg-1 ug/kg, 1 ug/kg-100 ug/kg, 100 ug/kg-500 ug/kg, 500 ug/kg-1 mg/kg, 1 mg/kg-50 mg/kg, 50 mg/kg-100 mg/kg, 100 mg/kg-500 mg/kg (body weight of recipient), although a lower or higher dosage may be administered. Dosages as low as about 1.0 mg/kg may be expected to show some efficacy. In some embodiments, about 5 mg/kg is an acceptable dosage, although dosage levels up to about 50 mg/kg are also preferred especially for therapeutic use. Alternatively, administration of a specific amount of the antibody may be given which is not based upon the weight of the patient such as an amount in the range of 1 ug-100 ug, 1 mg-100 mg, or 1 gm-100 gm. For example, site specific administration may be to body compartment or cavity such as intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, vaginal, rectal, buccal, sublingual, intranasal, or transdermal means.

The antibody compositions described herein can be prepared for use for parenteral (subcutaneous, intramuscular or intravenous) or any other administration particularly in the form of liquid solutions or suspensions. The formulation can also be suitable for an injectable formulation. In some embodiments, the injectable formulation is sterile. In some embodiments, the injectable formulation is pyrogen free. In some embodiments, the formulation is free of other antibodies that bind to other antigens other than an antigen described herein.

An amount is said to be sufficient or a “therapeutically effective amount” to “affect” the reduction of symptoms if the dosage, route of administration, and dosing schedule of the agent are sufficient to influence such a response. Responses to antibody administration can be measured by analysis of subject’s affected tissues, organs, or cells as by imaging techniques or by ex vivo analysis of tissue samples. An agent is physiologically significant if its presence results in a detectable change in the physiology of a recipient patient.

The antibodies can be formulated according to known methods to prepare pharmaceutically useful compositions, whereby these materials, or their functional derivatives, are combined in admixture with a pharmaceutically acceptable carrier vehicle. The treatment may be given in a single dose schedule, or a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease. In some embodiments, the antibody is administered weekly, once every two weeks, or once every 3 weeks, or once a month.

Kits are also provided which are useful for carrying out embodiments described herein. The present kits comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

In some embodiments, the following embodiments are provided:

1. A method of producing a population of nucleic acid molecules encoding a chicken complementary determining region (CDR) flanked by two human framework regions (FRs), the method comprising:

a) amplifying a first population of nucleic acid molecules encoding chicken antibodies with a first primer and a second primer under conditions sufficient to produce the amplified population of nucleic acid molecules encoding the chicken complementary determining region (CDR), wherein:
the first primer anneals to a region upstream or downstream of a CDR of the chicken antibody, wherein the first primer contains a restriction enzyme recognition site recognized by a restriction enzyme that cleaves at a location that is immediately upstream or downstream of the CDR and at a distance away from the recognition site; and
the second primer anneals to a region at a distance downstream of the CDR of the chicken antibody, if the first primer anneal upstream of the CDR; or
the second primer anneals to a region at a distance upstream of the CDR of the chicken antibody, if the first primer anneals downstream of the CDR.

2. The method of embodiment 1, further comprising digesting the amplified population of nucleic acid molecules with said restriction enzyme to produce a 5′ overhang immediately upstream of the sequence encoding the CDR to produce a digestion product.

3. The method of embodiment 2, further comprising preparing a first ligation product by ligating said digestion product to a nucleic acid sequence encoding a first FR of a human antibody,

wherein said first FR of a human antibody comprises an overhang region at its 3′ -end compatible with the overhang region of said digestion product so that the first FR is ligated to the upstream of the first digestion product.

4. The method of embodiment 1, wherein the restriction enzyme cleaves at a location that is 1, 2, 3, 4, or 5 nucleotides of the CDR boundary, wherein the boundary is either internal or external of the sequencing encoding the CDR.

5. The method of embodiment 4, wherein the restriction enzyme cleaves at a location that is 1, 2, or 3 nucleotides of the CDR boundary.

6. The method of embodiment 1, wherein the restriction enzyme cleaves at a location that is at least 10 nucleotides away from the recognition site.

7. The method of embodiment 1, wherein the restriction enzyme cleaves at a location that is 1, 2, 3, 4, or 5 nucleotides of the CDR boundary and at least 10 nucleotides away from the recognition site.

8. The method of embodiment 7, wherein the restriction enzyme cleaves at a location that is 1, 2, or 3 nucleotides of the CDR boundary and at least 10 nucleotides away from the recognition site.

9. The method of embodiment 1, wherein the second primer anneals to a region at a distance no greater than the length of the mRNA transcript downstream or upstream of the CDR of the chicken antibody.

10. The method of embodiment 2, wherein the 5′ overhang that is immediately downstream is 1, 2, 3, 4, or 5 nucleotides of the CDR boundary.

11. The method of embodiment 10, wherein the 5′ overhang that is immediately downstream is 1, 2, or 3 nucleotides of the CDR boundary.

12. The method of embodiment 3, the method further comprising preparing a second population of amplified nucleic acid molecules with a third primer and a fourth primer under conditions sufficient to produce the second amplified population of nucleic acid molecules encoding the chicken CDR, wherein:
the third primer anneals to a region immediately downstream of the nucleic acid sequence encoding the CDR present in the first ligation product, wherein the third primer contains a restriction enzyme recognition site recognized by a restriction enzyme that cleaves at a location that is immediately downstream of the CDR and at a distance away from the recognition site; and
the fourth primer anneals to a portion of the nucleic acid molecule encoding the first FR present in the first ligation product at a distance upstream of the CDR in the ligation product.

13. The method of embodiment 12, wherein the restriction enzyme cleaves at a location that is 1, 2, 3, 4, or 5 nucleotides of the CDR boundary.

14. The method of embodiment 13, wherein the restriction enzyme cleaves at a location that is 1, 2, or 3 nucleotides of the CDR boundary.

15. The method of embodiment 12, wherein the restriction enzyme cleaves at a location that is at least 10 nucleotides away from the recognition site.

16. The method of embodiment 12, wherein the restriction enzyme cleaves at a location that is 1, 2, 3, 4, or 5 nucleotides of the CDR boundary and at least 10 nucleotides away from the recognition site.

17. The method of embodiment 16, wherein the restriction enzyme cleaves at a location that is 1, 2, or 3 nucleotides of the CDR boundary and at least 10 nucleotides away from the recognition site.

18. The method of embodiment 12, wherein the second primer anneals to a region at a distance no greater than the length of the mRNA transcript downstream of the CDR of the chicken antibody.

19. The method of embodiment 12, further comprising digesting the second amplified population of nucleic acid molecules with said restriction enzyme to produce a 5′ overhang immediately downstream of the sequence encoding the CDR to produce a second digestion product.

20. The method of embodiment 19, wherein the 5′ overhang that is immediately downstream is 1, 2, 3, 4, or 5 nucleotides of the CDR boundary.

21. The method of embodiment 20, wherein the 5′ overhang that is immediately downstream is 1, 2, or 3 nucleotides of the CDR boundary.

22. The method of embodiment 19, further comprising ligating the second digestion product to a nucleic acid sequence encoding a second FR of a human antibody to produce the second ligation product, wherein said second FR comprises an overhang region at its 5′-end compatible with the overhang region of said second digestion product, so that the second FR is ligated to the downstream of the second digestion product.

23. The method of embodiment 22, further comprising amplifying the second ligation product with a first FW primer that anneals to the first FR of the human antibody and a second primer that anneals to the second FR of the human antibody under conditions sufficient to produce the population of nucleic acid molecules encoding the CDR flanked by two FRs of a human antibody.

24. The method of any one of embodiments 1-23, wherein the CDR is a chicken CDR1, CDR2, or CDR3.

25. The method of any one of embodiments 1-24, wherein the FRs are human FR1, FR2, FR3, or FR4.

26. The method of embodiment 25, wherein the first FR is human FR1 and the second FR is human FR2 .

27. The method of embodiment 25, wherein the first FR is human FR2 and the second FR is human FR3.

28. The method of embodiment 25, wherein the first FR is human FR3 and the second FR is human FR4.

29. The method of any one of embodiments 1-28, wherein the restriction enzyme is a Type IIS enzyme.

30. The method of embodiment 29, wherein the Type IIS enzyme is any Type IIS enzyme that cleaves at a distance greater than at least 10 bases away from the recognition sequence.

31. The method of embodiment 30, wherein the Type IIS enzyme cleaves at a distance of 14-21 bases away from the recognition sequence.

32. The method of embodiment 31, wherein the Type IIS enzyme is AcuI, BpmI, BpuEI, BsgI, MmeI, or NmeAllI.

33. The method of any one of embodiments 1-32, wherein the method further comprises producing a nucleic acid encoding a protein comprising a chicken CDR1, a chicken CDR2, and a chicken CDR3, each CDR flanked by a human framework region.

34. The method of embodiment 33, wherein the producing the nucleic acid encoding a protein comprising a chicken CDR1, a chicken CDR2, and a chicken CDR3, each CDR flanked by a human framework region comprises performing overlapping PCR of a first, second, and third ligation product produced according to the methods of any one of embodiments 1-32, wherein:
the first ligation product comprises a nucleic acid molecule encoding a chicken CDR1 flanked by a human framework region;
the second ligation product comprises a nucleic acid molecule encoding a chicken CDR2 flanked by a human framework region; and
the third ligation product comprises a nucleic acid molecule encoding a chicken CDR3 flanked by a human framework region.

35. The method of embodiment 33, wherein the producing the nucleic acid encoding a protein comprising a chicken CDR1, a chicken CDR2, and a chicken CDR3, each CDR flanked by a human framework region comprises ligating a first, second, and third ligation product produced according to the methods of any one of embodiments 1-32, wherein:
the first ligation product comprises a nucleic acid molecule encoding a chicken CDR1 flanked by a human framework region;
the second ligation product comprises a nucleic acid molecule encoding a chicken CDR2 flanked by a human framework region; and
the third ligation product comprises a nucleic acid molecule encoding a chicken CDR3 flanked by a human framework region.

36. The method of any one of embodiments 33-35, wherein the nucleic acid molecule encoding a protein comprising a chicken CDR1, a chicken CDR2, and a chicken CDR3 has a formula of:

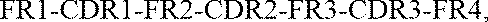

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

37. The method of any one of embodiment 33-35, wherein the nucleic acid molecule encoding a protein comprising a chicken CDR1, a chicken CDR2, and a chicken CDR3 encodes an antibody variable region.

38. The method of any one of embodiments 1-37, wherein the first primer and third primer comprise a nucleic acid sequence comprising the formula:

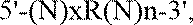

wherein,
R is the recognition sequence;
N is any nucleic acid base, such as naturally occurring, non-naturally occurring, or degenerate nucleic acid base;
x is 0-11; and
n is 1-21.

39. The method of embodiment 38, wherein R is CTGAAG, CGATC, ACNNNNGTAYC, GAAGAC, GCAGC, CCATC, ACGGC, CGANNNNNNTGC, GTATCC, GTCTC, ACCTGC, ACTGGG, CTGGAG, CTTGAG, GGTCTC, ACNNNNNCTCC, GAGGAG, GTGCAG, GTCTC, CGTCTC, GGGAC, GAATGC, CTCAG, ACCTGC, GCTCTTC, GCAATG, ACTGG, GCGATG, GGATG, GCAGTG, CAGTG, CAANNNNNGTGG, CTCTTC, GGCGGA, CGTCTC, CCCGC, GGATG, GACGC, GGTGA, CCTTC, GAAGA, GAGTC, TCCRAC, CCTC, GCCGAG, GAGTC, GCTCTTC, or GCATC.

40. The method of embodiment 38, wherein the naturally occurring nucleic acid base is C, G, T, A, or U.

41. The method of embodiment 38, wherein the non-naturally occurring nucleic acid base stabilizes the DNA duplex.

43. The method of embodiment 41, wherein the non-naturally occurring nucleic acid base is AP-dC, 2-Aminoadenine, 5-methylcystosine, C(5)-propynylcystosine, or C(5)-propynyluracil.

43. The method of embodiment 38, wherein the degenerate nucleic acid base is K, M, R, Y, S, W, B, D or H.

44. The method of embodiment 43, wherein K is G or T/U.

45. The method of embodiment 43, wherein M is A or C.

46. The method of embodiment 43, wherein R is A or G.

47. The method of embodiment 43, wherein Y is C or T/U.

48. The method of embodiment 43, wherein S is C or G.

49. The method of embodiment 43, wherein W is A or T/U.

50. The method of embodiment 43, wherein B is C or G or T/U.

51. The method of embodiment 43, wherein D is A or G or T/U.

52. The method of embodiment 43, wherein H is A or C or T/U.

53. The method of any one of embodiments 1-52, wherein the first primer and third primer comprise a nucleic acid sequence comprises a sequence of 5′-CTGAAGNNNNNNNNNNNNNNNN-3′ (SEQ ID NO: 45), wherein N is any nucleic acid base, such as naturally occurring, non-naturally occurring, or degenerate nucleic acid base.

54. The method of embodiment 53, wherein the naturally occurring nucleic acid base is C, G, T, A, or U.

55. The method of embodiment 53, wherein the non-naturally occurring nucleic acid base stabilizes the DNA duplex.

56. The method of embodiment 55, wherein the non-naturally occurring nucleic acid base is AP-dC, 2-Aminoadenine, 5-methylcystosine, C(5)-propynylcystosine, or C(5)-propynyluracil.

57. The method of embodiment 53, wherein the degenerate nucleic acid base is K, M, R, Y, S, W, B, D or H.

58. The method of embodiment 57, wherein K is G or T/U.

59. The method of embodiment 57, wherein M is A or C.

60. The method of embodiment 57, wherein R is A or G.

61. The method of embodiment 57, wherein Y is C or T/U.

62. The method of embodiment 57, wherein S is C or G.

63. The method of embodiment 57, wherein W is A or T/U.

64. The method of embodiment 57, wherein B is C or G or T/U.

65. The method of embodiment 57, wherein D is A or G or T/U.

66. The method of embodiment 57, wherein H is A or C or T/U.

67. The method of any one of embodiments 1-52, wherein the first primer and third primer comprise a nucleic acid sequence comprises a sequence of 5′-CTGGAGNNNNNNNNNNNNNNNN-3′, wherein N is any nucleic acid base, such as naturally occurring, non-naturally occurring, or degenerate nucleic acid base.

68. The method of embodiment 67, wherein the naturally occurring nucleic acid base is C, G, T, A, or U.

69. The method of embodiment 67, wherein the non-naturally occurring nucleic acid base stabilizes the DNA duplex.

70. The method of embodiment 69, wherein the non-naturally occurring nucleic acid base is AP-dC, 2-Aminoadenine, 5-methylcystosine, C(5)-propynylcystosine, or C(5)-propynyluracil.

71. The method of embodiment 67, wherein the degenerate nucleic acid base is K, M, R, Y, S, W, B, D or H.

72. The method of embodiment 71, wherein K is G or T/U.

73. The method of embodiment 71, wherein M is A or C.

74. The method of embodiment 71, wherein R is A or G.

75. The method of embodiment 71, wherein Y is C or T/U.

76. The method of embodiment 71, wherein S is C or G.

77. The method of embodiment 71, wherein W is A or T/U.

78. The method of embodiment 71, wherein B is C or G or T/U.

79. The method of embodiment 71, wherein D is A or G or T/U.

80. The method of embodiment 71, wherein H is A or C or T/U.

81. The method of any one of embodiments 1-52, wherein the first primer and third primer comprise a nucleic acid sequence comprises a sequence of 5′-CTTGAGNNNNNNNNNNNNNNNN-3′, wherein N is any nucleic acid base, such as naturally occurring, non-naturally occurring, or degenerate nucleic acid base.

82. The method of embodiment 81, wherein the naturally occurring nucleic acid base is C, G, T, A, or U.

83. The method of embodiment 81, wherein the non-naturally occurring nucleic acid base stabilizes the DNA duplex.

84. The method of embodiment 83, wherein the non-naturally occurring nucleic acid base is AP-dC, 2-Aminoadenine, 5-methylcystosine, C(5)-propynylcystosine, or C(5)-propynyluracil.

85. The method of embodiment 81, wherein the degenerate nucleic acid base is K, M, R, Y, S, W, B, D or H.

86. The method of embodiment 85, wherein K is G or T/U.

87. The method of embodiment 85, wherein M is A or C.

88. The method of embodiment 85, wherein R is A or G.

89. The method of embodiment 85, wherein Y is C or T/U.

90. The method of embodiment 85, wherein S is C or G.

91. The method of embodiment 85, wherein W is A or T/U.

92. The method of embodiment 85, wherein B is C or G or T/U.

93. The method of embodiment 85, wherein D is A or G or T/U.

94. The method of embodiment 85, wherein H is A or C or T/U.

95. The method of any one of embodiments 1-52, wherein the first primer and third primer comprise a nucleic acid sequence comprises a sequence of 5′-GTGCAGNNNNNNNNNNNNNNNN-3′ (SEQ ID NO: 62), wherein N is any nucleic acid base, such as naturally occurring, non-naturally occurring, or degenerate nucleic acid base.

96. The method of embodiment 95, wherein the naturally occurring nucleic acid base is C, G, T, A, or U.

97. The method of embodiment 95, wherein the non-naturally occurring nucleic acid base stabilizes the DNA duplex.

98. The method of embodiment 97, wherein the non-naturally occurring nucleic acid base is AP-dC, 2-Aminoadenine, 5-methylcystosine, C(5)-propynylcystosine, or C(5)-propynyluracil.

99. The method of embodiment 95, wherein the degenerate nucleic acid base is K, M, R, Y, S, W, B, D or H.

100. The method of embodiment 99, wherein K is G or T/U.

101. The method of embodiment 99, wherein M is A or C.

102. The method of embodiment 99, wherein R is A or G.

103. The method of embodiment 99, wherein Y is C or T/U.

104. The method of embodiment 99, wherein S is C or G.

105. The method of embodiment 99, wherein W is A or T/U.

106. The method of embodiment 99, wherein B is C or G or T/U.

107. The method of embodiment 99, wherein D is A or G or T/U.

108. The method of embodiment 99, wherein H is A or C or T/U.

109. The method of any one of embodiments 1-52, wherein the first primer and third primer comprise a nucleic acid sequence comprises a sequence of 5′-TCCRACNNNNNNNNNNNNNNNNNNNN-3′, wherein N is any nucleic acid base, such as naturally occurring, non-naturally occurring, or degenerate nucleic acid base.

110. The method of embodiment 109, wherein the naturally occurring nucleic acid base is C, G, T, A, or U.

111. The method of embodiment 109, wherein the non-naturally occurring nucleic acid base stabilizes the DNA duplex.

112. The method of embodiment 111, wherein the non-naturally occurring nucleic acid base is AP-dC, 2-Aminoadenine, 5-methylcystosine, C(5)-propynylcystosine, or C(5)-propynyluracil.

113. The method of embodiment 109, wherein the degenerate nucleic acid base is K, M, R, Y, S, W, B, D or H.

114. The method of embodiment 113, wherein K is G or T/U.

115. The method of embodiment 113, wherein M is A or C.

116. The method of embodiment 113, wherein R is A or G.

117. The method of embodiment 113, wherein Y is C or T/U.

118. The method of embodiment 113, wherein S is C or G.

119. The method of embodiment 113, wherein W is A or T/U.

120. The method of embodiment 113, wherein B is C or G or T/U.

121. The method of embodiment 113, wherein D is A or G or T/U.

122. The method of embodiment 113, wherein H is A or C or T/U.

123. The method of any one of embodiments 1-52, wherein the first primer and third primer comprise a nucleic acid sequence comprises a sequence of 5′-GCCGAGNNNNNNNNNNNNNNNNNNNNN-3′, wherein N is any nucleic acid base, such as naturally occurring, non-naturally occurring, or degenerate nucleic acid base.

124. The method of embodiment 123, wherein the naturally occurring nucleic acid base is C, G, T, A, or U.

125. The method of embodiment 123, wherein the non-naturally occurring nucleic acid base stabilizes the DNA duplex.

126. The method of embodiment 125, wherein the non-naturally occurring nucleic acid base is AP-dC, 2-Aminoadenine, 5-methylcystosine, C(5)-propynylcystosine, or C(5)-propynyluracil.

127. The method of embodiment 123, wherein the degenerate nucleic acid base is K, M, R, Y, S, W, B, D or H.

128. The method of embodiment 127, wherein K is G or T/U.

129. The method of embodiment 127, wherein M is A or C.

130. The method of embodiment 127, wherein R is A or G.

131. The method of embodiment 127, wherein Y is C or T/U.

132. The method of embodiment 127, wherein S is C or G.

133. The method of embodiment 127, wherein W is A or T/U.

134. The method of embodiment 127, wherein B is C or G or T/U.

135. The method of embodiment 127, wherein D is A or G or T/U.

136. The method of embodiment 127, wherein H is A or C or T/U.

137. A method of producing a library of nucleic acid molecules encoding humanized variable regions of antibodies, the method comprising combining:
i) a first library of nucleic acid molecules encoding a chicken complementary determining region 1 (CDR1) domain, flanked by nucleic acid sequences encoding a human framework region 1 (FR1) and a human framework region 2 (FR2);
ii) a second library of nucleic acid sequences encoding a chicken complementary determining region 2 (CDR2) domain, flanked by nucleic acid sequences encoding a human framework region 2 (FR2) and a human framework region 3 (FR3);
iii) a third library of nucleic acid sequences encoding a chicken complementary determining region 3 (CDR3) domain, flanked by nucleic acid sequences encoding a human framework region 3 (FR3) and a human framework region 4 (FR4),
   wherein the nucleic acid molecule encoding the humanized variable regions of antibodies has a formula of: 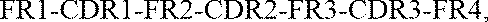
   wherein
      FR1 is a human FR1;
      CDR1 is a chicken CDR1;
      FR2 is a human FR2;
      CDR2 is a chicken CDR2;
      FR3 is a human FR3;
      CDR3 is a chicken CDR3; and
      FR4 is a human FR4.

138. The method of embodiment 137, wherein the combining comprises ligating the first, second, and third libraries of nucleic acid molecules to produce the nucleic acid molecule encoding the humanized variable regions of antibodies.

139. The method of embodiment 137, wherein the combining comprises performing overlapping PCR with the first, second, and third libraries of nucleic acid molecules to produce the nucleic acid molecule encoding the humanized variable regions of antibodies.

140. A library of nucleic acid molecules prepared according to any one of embodiments 1-139.

141. The library of nucleic acid molecules of embodiment 140, wherein the library comprises a population of nucleic acid molecules, a plurality of nucleic acid molecules of the population encodes a polypeptide having a formula of:

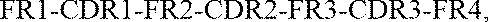

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

142. The library of embodiment 141, wherein there are at least two nucleic acid molecules present in the library that encode different polypeptides.

143. An oligonucleotide that anneals to a region immediately upstream or downstream of CDR of a chicken antibody, and wherein said oligonucleotide comprises a restriction enzyme recognition site recognized by a restriction enzyme that cleaves at a distance downstream of the recognition site.

144. The oligonucleotide of embodiment 143, wherein the oligonucleotide comprises the sequence of nucleic acid sequence comprising the formula:

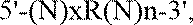

wherein,
R is the recognition sequence;
N is any nucleic acid base, such as naturally occurring, non-naturally occurring, or degenerate nucleic acid base;
x is 0-11; and
n is 1-21.

145. The oligonucleotide of embodiment 144, wherein R is CTGAAG, CGATC, ACNNNNGTAYC, GAAGAC, GCAGC, CCATC, ACGGC, CGANNNNNNTGC, GTATCC, GTCTC, ACCTGC, ACTGGG, CTGGAG, CTTGAG, GGTCTC, ACNNNNNCTCC, GAGGAG, GTGCAG, GTCTC, CGTCTC, GGGAC, GAATGC, CTCAG, ACCTGC, GCTCTTC, GCAATG, ACTGG, GCGATG, GGATG, GCAGTG, CAGTG, CAANNNNNGTGG, CTCTTC, GGCGGA, CGTCTC, CCCGC, GGATG, GACGC, GGTGA, CCTTC, GAAGA, GAGTC, TCCRAC, CCTC, GCCGAG, GAGTC, GCTCTTC, or GCATC.

146. The oligonucleotide of embodiment 144, wherein the naturally occurring nucleic acid base is C, G, T, A, or U.

147. The oligonucleotide of embodiment 144, wherein the non-naturally occurring nucleic acid base stabilizes the DNA duplex.

148. The oligonucleotide of embodiment 147, wherein the non-naturally occurring nucleic acid base is AP-dC, 2-Aminoadenine, 5-methylcystosine, C(5)-propynylcystosine, or C(5)-propynyluracil.

149. The oligonucleotide of embodiment 144, wherein the degenerate nucleic acid base is K, M, R, Y, S, W, B, D or H.

150. The oligonucleotide of embodiment 149, wherein K is G or T/U.

151. The oligonucleotide of embodiment 149, wherein M is A or C.

152. The oligonucleotide of embodiment 149, wherein R is A or G.

153. The oligonucleotide of embodiment 149, wherein Y is C or T/U.

154. The oligonucleotide of embodiment 149, wherein S is C or G.

155. The oligonucleotide of embodiment 149, wherein W is A or T/U.

156. The oligonucleotide of embodiment 149, wherein B is C or G or T/U.

157. The oligonucleotide of embodiment 149, wherein D is A or G or T/U.

158. The oligonucleotide of embodiment 149, wherein H is A or C or T/U.

159. The oligonucleotide of embodiment 143, wherein the oligonucleotide comprises the sequence of nucleic acid sequence comprises a sequence of 5′-CTGAAGNNNNNNNNNNNNNNNN-3′ (SEQ ID NO: 45), wherein N is any nucleic acid base, such as naturally occurring, non-naturally occurring, or degenerate nucleic acid base.

160. The oligonucleotide of embodiment 159, wherein the naturally occurring nucleic acid base is C, G, T, A, or U.

161. The oligonucleotide of embodiment 159, wherein the non-naturally occurring nucleic acid base stabilizes the DNA duplex.

162. The oligonucleotide of embodiment 161, wherein the non-naturally occurring nucleic acid base is AP-dC, 2-Aminoadenine, 5-methylcystosine, C(5)-propynylcystosine, or C(5)-propynyluracil.

163. The oligonucleotide of embodiment 159, wherein the degenerate nucleic acid base is K, M, R, Y, S, W, B, D or H.

164. The oligonucleotide of embodiment 163, wherein K is G or T/U.

165. The oligonucleotide of embodiment 163, wherein M is A or C.

166. The oligonucleotide of embodiment 163, wherein R is A or G.

167. The oligonucleotide of embodiment 163, wherein Y is C or T/U.

168. The oligonucleotide of embodiment 163, wherein S is C or G.

169. The oligonucleotide of embodiment 163, wherein W is A or T/U.

170. The oligonucleotide of embodiment 163, wherein B is C or G or T/U.

171. The oligonucleotide of embodiment 163, wherein D is A or G or T/U.

172. The oligonucleotide of embodiment 163, wherein H is A or C or T/U.

173. The oligonucleotide of embodiment 143, wherein the oligonucleotide comprises the sequence of nucleic acid sequence comprises a sequence of 5′-CTGGAGNNNNNNNNNNNNNNNN-3′, wherein N is any nucleic acid base, such as naturally occurring, non-naturally occurring, or degenerate nucleic acid base.

174. The oligonucleotide of embodiment 173, wherein the naturally occurring nucleic acid base is C, G, T, A, or U.

175. The oligonucleotide of embodiment 173, wherein the non-naturally occurring nucleic acid base stabilizes the DNA duplex.

176. The oligonucleotide of embodiment 175, wherein the non-naturally occurring nucleic acid base is AP-dC, 2-Aminoadenine, 5-methylcystosine, C(5)-propynylcystosine, or C(5)-propynyluracil.

177. The oligonucleotide of embodiment 173, wherein the degenerate nucleic acid base is K, M, R, Y, S, W, B, D or H.

178. The oligonucleotide of embodiment 177, wherein K is G or T/U.

179. The oligonucleotide of embodiment 177, wherein M is A or C.

180. The oligonucleotide of embodiment 177, wherein R is A or G.

181. The oligonucleotide of embodiment 177, wherein Y is C or T/U.

182. The oligonucleotide of embodiment 177, wherein S is C or G.

183. The oligonucleotide of embodiment 177, wherein W is A or T/U.

184. The oligonucleotide of embodiment 177, wherein B is C or G or T/U.

185. The oligonucleotide of embodiment 177, wherein D is A or G or T/U.

186. The oligonucleotide of embodiment 177, wherein H is A or C or T/U.

187. The oligonucleotide of embodiment 143, wherein the oligonucleotide comprises the sequence of nucleic acid sequence comprises a sequence of 5′-CTTGAGNNNNNNNNNNNNNNNN-3′, wherein N is any nucleic acid base, such as naturally occurring, non-naturally occurring, or degenerate nucleic acid base.

188. The oligonucleotide of embodiment 187, wherein the naturally occurring nucleic acid base is C, G, T, A, or U.

189. The oligonucleotide of embodiment 187, wherein the non-naturally occurring nucleic acid base stabilizes the DNA duplex.

190. The oligonucleotide of embodiment 189, wherein the non-naturally occurring nucleic acid base is AP-dC, 2-Aminoadenine, 5-methylcystosine, C(5)-propynylcystosine, or C(5)-propynyluracil.

191. The oligonucleotide of embodiment 187, wherein the degenerate nucleic acid base is K, M, R, Y, S, W, B, D or H.

192. The oligonucleotide of embodiment 191, wherein K is G or T/U.

193. The oligonucleotide of embodiment 191, wherein M is A or C.

194. The oligonucleotide of embodiment 191, wherein R is A or G.

195. The oligonucleotide of embodiment 191, wherein Y is C or T/U.

196. The oligonucleotide of embodiment 191, wherein S is C or G.

197. The oligonucleotide of embodiment 191, wherein W is A or T/U.

198. The oligonucleotide of embodiment 191, wherein B is C or G or T/U.

199. The oligonucleotide of embodiment 191, wherein D is A or G or T/U.

200. The oligonucleotide of embodiment 191, wherein H is A or C or T/U.

201. The oligonucleotide of embodiment 143, wherein the oligonucleotide comprises the sequence of nucleic acid sequence comprises a sequence of 5′-GTGCAGNNNNNNNNNNNNNNNN-3′ (SEQ ID NO: 62), wherein N is any nucleic acid base, such as naturally occurring, non-naturally occurring, or degenerate nucleic acid base.

202. The oligonucleotide of embodiment 201, wherein the naturally occurring nucleic acid base is C, G, T, A, or U.

203. The oligonucleotide of embodiment 201, wherein the non-naturally occurring nucleic acid base stabilizes the DNA duplex.

204. The oligonucleotide of embodiment 203, wherein the non-naturally occurring nucleic acid base is AP-dC, 2-Aminoadenine, 5-methylcystosine, C(5)-propynylcystosine, or C(5)-propynyluracil.

205. The oligonucleotide of embodiment 201, wherein the degenerate nucleic acid base is K, M, R, Y, S, W, B, D or H.

206. The oligonucleotide of embodiment 205, wherein K is G or T/U.

207. The oligonucleotide of embodiment 205, wherein M is A or C.

208. The oligonucleotide of embodiment 205, wherein R is A or G.

209. The oligonucleotide of embodiment 205, wherein Y is C or T/U.

210. The oligonucleotide of embodiment 205, wherein S is C or G.

211. The oligonucleotide of embodiment 205, wherein W is A or T/U.

212. The oligonucleotide of embodiment 205, wherein B is C or G or T/U.

213. The oligonucleotide of embodiment 205, wherein D is A or G or T/U.

214. The oligonucleotide of embodiment 205, wherein H is A or C or T/U.

215. The oligonucleotide of embodiment 143, wherein the oligonucleotide comprises the sequence of nucleic acid sequence comprises a sequence of 5′-TCCRACNNNNNNNNNNNNNNNNNNNN-3′, wherein N is any nucleic acid base, such as naturally occurring, non-naturally occurring, or degenerate nucleic acid base.

216. The oligonucleotide of embodiment 215, wherein the naturally occurring nucleic acid base is C, G, T, A, or U.

217. The oligonucleotide of embodiment 215, wherein the non-naturally occurring nucleic acid base stabilizes the DNA duplex.

218. The oligonucleotide of embodiment 217, wherein the non-naturally occurring nucleic acid base is AP-dC, 2-Aminoadenine, 5-methylcystosine, C(5)-propynylcystosine, or C(5)-propynyluracil.

219. The oligonucleotide of embodiment 215, wherein the degenerate nucleic acid base is K, M, R, Y, S, W, B, D or H.

220. The oligonucleotide of embodiment 219, wherein K is G or T/U.

221. The oligonucleotide of embodiment 219, wherein M is A or C.

222. The oligonucleotide of embodiment 219, wherein R is A or G.

223. The oligonucleotide of embodiment 219, wherein Y is C or T/U.

224. The oligonucleotide of embodiment 219, wherein S is C or G.

225. The oligonucleotide of embodiment 219, wherein W is A or T/U.

226. The oligonucleotide of embodiment 219, wherein B is C or G or T/U.

227. The oligonucleotide of embodiment 219, wherein D is A or G or T/U.

228. The oligonucleotide of embodiment 219, wherein H is A or C or T/U.

229. The oligonucleotide of embodiment 143, wherein the oligonucleotide comprises the sequence of nucleic acid sequence comprises a sequence of 5′-GCCGAGNNNNNNNNNNNNNNNNNNNNN-3′, wherein N is any nucleic acid base, such as naturally occurring, non-naturally occurring, or degenerate nucleic acid base.

230. The oligonucleotide of embodiment 229, wherein the naturally occurring nucleic acid base is C, G, T, A, or U.

231. The oligonucleotide of embodiment 229, wherein the non-naturally occurring nucleic acid base stabilizes the DNA duplex.

232. The oligonucleotide of embodiment 231, wherein the non-naturally occurring nucleic acid base is AP-dC, 2-Aminoadenine, 5-methylcystosine, C(5)-propynylcystosine, or C(5)-propynyluracil.

233. The oligonucleotide of embodiment 229, wherein the degenerate nucleic acid base is K, M, R, Y, S, W, B, D or H.

234. The oligonucleotide of embodiment 233, wherein K is G or T/U.

235. The oligonucleotide of embodiment 233, wherein M is A or C.

236. The oligonucleotide of embodiment 233, wherein R is A or G.

237. The oligonucleotide of embodiment 233, wherein Y is C or T/U.

238. The oligonucleotide of embodiment 233, wherein S is C or G.

239. The oligonucleotide of embodiment 233, wherein W is A or T/U.

240. The oligonucleotide of embodiment 233, wherein B is C or G or T/U.

241. The oligonucleotide of embodiment 233, wherein D is A or G or T/U.

242. The oligonucleotide of embodiment 233, wherein H is A or C or T/U.

243. An oligonucleotide as provided or described herein.

244. A polypeptide encoded by a nucleic acid molecule having a formula of:

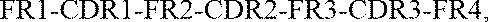

wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4,
wherein the nucleic acid molecule was prepared according to any one of embodiments 1-139.

245. A method of identifying a binding partner to a target, the method comprising contacting the target with a library of proteins encoded by a library prepared according to a method of any one of embodiments 1-139.

246. The method of embodiment 245, wherein the binding partner is an antibody.

247. The method of embodiment 245, wherein the binding partner is a scFv.

Although the embodiments have been described with respect to various embodiments, it is not intended to be limited thereto, but rather those skilled in the art will recognize that variations and modifications may be made therein which are within the spirit of the embodiments and the scope thereof.

Example 1: Chicken VH Library Humanization: This example presents a nonlimiting method for extraction of VH CDR2 and CDR3 from B cells of an immunized chicken, followed by assembly with human framework regions to form a humanized VH coding region containing chicken CDRs.

Human framework region acceptor fragments were generated by PCR amplification from a plasmid containing a germline human V-gene, followed by digestion with a restriction enzyme to leave an overhang compatible with extracted chicken CDRs. VH3-23 was chosen due to similarity at the amino acid level to the chicken V-gene. The sequence of VH3-23 V-gene in addition to human JH1 (FR4) (SEQ ID 1) are shown in Table 1. The VH3-23 V-gene plus JH1 were codon optimized in order to ensure identity with the chicken codons at planned junction points and inserted into a pUC vector for use as a template for framework amplification.

TABLE 1

Nucleic acid sequence of VH3-23 V-gene and human JH1 (SEQ ID NO: 1)
CGAGGTGCAGCTGCTCGAATCCGGTGGTGGCCTGGTCCAACCTGGCGGCAGCCTG CGCCTGTCTTGCGCTGCCTCCGGCTTTACCTTCTCCAGCTACGCCATGTCCTGGGTG CGCCAAGCTCCTGGTAAAGGCCTGGAATGGGTCAGCGCTATCAGCGGTTCTGGCG GCTCCACCTATTACGCTGATTCCGTGAAAGGCCGCTTCACCATCAGCCGCGACAAC TCTAAGAATACACTGTATCTGCAAATGAACTCCCTCCGCGCCGAAGACACTGCAG TCTACTACTGCGCCAAAGGCTGGGGTCAAGGCACCCTGGTGACTGTCTCCAGCAC TAGTGGGGCCGGTGGGGCCCAGAATTCCAGCTGCG

The primers used to generate the human framework region acceptor fragments are listed in Table 2 (SEQ IDs 2-9). One primer in each primer pair contains an AcuI restriction enzyme recognition site (CTGAAG in Table 2) at such a position that the cleavage of the restriction enzyme would leave an overhang at the desired junction point for ligation to the extracted chicken CDRs. This primer is also biotinylated on the 5′ end for capture onto streptavidin magnetic beads and release by digestion with AcuI. A random spacer sequence between the biotin and AcuI recognition site is included to improve digestion efficiency. The random spacer sequence allows for efficient cleavage by AcuI and is limited in length by primer synthesis.

TABLE 2

Primer Sequences for Human Framework Region Acceptor Fragments.

| | | |
|---|---|---|
| Primer set A | GAGGTGCAGCTGCTCGAATCC | SEQ ID NO: 2 |
| | /5Biosg/CACATGCGGATCCGCAGTGGctgaagGGCTGATASCGCTGACCCATTC C | SEQ ID NO: 3 |
| Primer set B | CAGTAGTAGACTGCAGTGTCTTCGGCG | SEQ ID NO: 4 |
| | /5Biosg/CACATGCGGATCCGCAGTGGctgaagGCAGCTACACCTATTACGCTGA TTCCG (SEQ ID NO: 5) or /5Biosg/CACATGCGGATCCGCAGTGGctgaagATTACGCTGATTCCGTGAAAGG CC (SEQ ID NO: 43) | |
| Primer set C | GCTGATTCCGTGAAAGGCCG | SEQ ID NO: 6 |
| | /5Biosg/CACATGCGGATCCGCAGTGGctgaagCCCAGCCTTTGGCGCAG | SEQ ID NO: 7 |
| Primer set D | /5Biosg/CACATGCGGATCCGCAGTGGctgaagGCGCCAAAGGCTGGGGTC | SEQ ID NO: 8 |
| | CGCAGCTGGAATTCTGGGCC | SEQ ID NO: 9 |

PCR reactions were set up using 2X Phusion MasterMix (NEB), 1 ng template (SEQ ID 1), and 0.5 uM each primer using the following thermocycling conditions: 98° for 30 sec, 30 cycles of 98° for 5 sec, 72° for 15 sec, and a final cycle of 72° for 2 min. Following completion of thermocycling, reactions were purified using a PCR purification kit (Qiagen) and captured using 50 ul streptavidin-magnetic beads (NEB). Following capture, beads were washed with wash buffer (0.5 M NaCl, 20 mM Tris-HCl, 0.1 mM EDTA) followed by 1X Cutsmart Buffer (NEB). PCR product was released from the beads by setting up a restriction digestion reaction with AcuI (NEB), Cutsmart Buffer (NEB), and S-adenosylmethionine (NEB) and incubating at 37°, shaking at 1,500 rpm for 30 min in a thermomixer. Following digestion, the supernatant containing released PCR product was purified using a PCR purification kit (Qiagen).

The 4 human framework region acceptor fragments were generated and their sequences are listed in Table 3. The FR1-CDRH1-FR2 fragment (SEQ ID NO:10) contains the human VH3-23 germline CDR1 region (CDRH1) in addition to flanking frameworks FR1 and FR2. This fragment was generated using Primer Set A in Table 2. The FR3 (H2 overhang) fragment (SEQ ID NO: 11 or SEQ ID NO: 44) contains a single framework region FR3 with an overhang at the terminal where the FR3 region connects to the CDR2 region of the human VH3-23 gene. This fragment was generated using one of the Primer Sets B in Table 2. The FR3 (H3 overhang) fragment (SEQ ID NO: 12) contains a single framework region FR3 with an overhang at the terminal where the FR3 region connects to the CDR3 region of the human VH3-23 gene. This fragment was generated using Primer Set C in Table 2. The FR4 fragment (SEQ ID NO: 13) contains a single framework region FR4 and was generated using Primer Set D in Table 2.

TABLE 3

Human Framework Region Acceptor Fragments. The overhang regions generated by Acul are underlined.
Human Framework Region Acceptor Fragments

| | | |
|---|---|---|
| | 5′ | S E Q I D N O 1 0 S E Q I D N |
| FR1- | AGGTGCAGCTGCTCGAATCCGGTGGTGGCCTGGTCCAACCTGGCGGCAGCCTGCGCCTGTCTTGCGCTG CCTCCGGCTTTACCTTCTCCAGCTACGCCATGTCCTGGGTGCGCCAAGCTCCTGGTAAAGGCCTGGAAT GGGT | |
| CDRH | | |
| 1- | 3′ | |
| FR2 | TCCACGTCGACGAGCTTAGGCCACCACCGGACCAGGTTGGACCGCCGTCGGACGCGGACAGAACGCGAC GGAGGCCGAAATGGAAGAGGTCGATGCGGTACAGGACCCACGCGGTTCGAGGACCATTTCCGGACCTTA CC | |
| FR3 | 5′ | |
| (H2 | CGCTGATTCCGTGAAAGGCCGCTTCACCATCAGCCGCGACAACTCTAAGAATACACTGTATCTGCAAAT | |
| over | GAACTCCCTCCGCGCCGAAGACACTGCAGTCTACTACTG | |
| hang | 3′ | |
| ) | ATGCGACTAAGGCACTTTCCGGCGAAGTGGTAGTCGGCGCTGTTGAGATTCTTATGTGACATAGACGTT TACTTGAGGGAGGCGCGGCTTCTGTGACGTCAGATGATGAC | |
| FR3 (H2 over hang ) | 5′ GAAAGGCCGCTTCACCATCAGCCGCGACAACTCTAAGAATACACTGTATCTGCAAATGAACTCCCTCCG CGCCGAAGACACTGCAGTCTACTACTG 3′ | O : 1 1 S E Q I D N O |
| FR3 (H3 over hang ) | CACTTTCCGGCGAAGTGGTAGTCGGCGCTGTTGAGATTCTTATGTGACATAGACGTTTACTTGAGGGAG GCGCGGCTTCTGTGACGTCAGATGATGAC 5′ GCTGATTCCGTGAAAGGCCGCTTCACCATCAGCCGCGACAACTCTAAGAATACACTGTATCTGCAAATG AACTCCCTCCGCGCCGAAGACACTGCAGTCTACTACTG 3′ CGACTAAGGCACTTTCCGGCGAAGTGGTAGTCGGCGCTGTTGAGATTCTTATGTGACATAGACGTTTAC TTGAGGGAGGCGCGGCTTCTGTGACGTCAGATGATG | : 4 4 S E Q I D N O |
| FR4 | 5′ TCAAGGCACCCTGGTGACTGTCTCCAGCACTAGTGGGGCCGGTGGGGCCCAGAATTCCAGCTGCG 3′ CCAGTTCCGTGGGACCACTGACAGAGGTCGTGATCACCCCGGCCACCCCGGGTCTTAAGGTCGACGC | : 1 2 S E Q I D N O : 1 3 |

Obtaining the CDR2 region of a chicken antibody VH chain and insertion into the human framework. Chicken VH CDR2 was amplified from cDNA prepared from RNA extracted from B cells of an immunized chicken. The CDR2 fragment is extracted by several PCR and digestion reactions as described herein and above. The CDR2 fragment is then ligated onto the human framework region acceptor fragments, generating a fragment containing chicken CDR2 flanked by human framework regions.

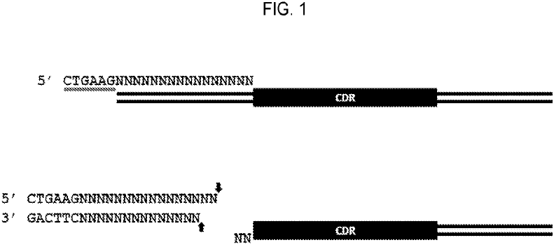

The primers used for CDR2 amplification are listed in Table 4 (SEQs ID No: 14-28). For each PCR step the primer set included a pool of primers that bind to chicken framework regions adjacent to the CDR. Without being bound to any particular theory, these pools of primers contain degenerate bases in order to sample as much of the chicken immune repertoire as possible. These primers contain an AcuI restriction enzyme recognition site (CTGAAG in Table 4) positioned such that the cleavage of the restriction enzyme leaves an overhang at the desired junction point for ligation to the human framework region acceptor fragments. These primers are biotinylated on the 5′ end for capture onto streptavidin magnetic beads and release by digestion with AcuI. A random spacer sequence between the biotin and AcuI recognition site is included to improve digestion efficiency. The design of a primer containing AcuI recognition site is shown in FIG. 1.

TABLE 4

Primer Sequences for Chicken VH CDR2 Amplification

| | |
|---|---|
| GCCGTGACGTTGGACGAG | SEQ ID NO: 14 |
| /5Biosg/CACATGCGGATCCGCAGTGGctgaagTCACCGCCGVCCCGTA | SEQ ID NO: 15 |
| /5Biosg/CACATGCGGATCCGCAGTGGctgaagTCACCGCCGVCSCGTA | SEQ ID NO: 16 |
| /5Biosg/CACATGCGGATCCGCAGTGGctgaagTCACCGCCGVCSCRTA | SEQ ID NO: 17 |
| /5Biosg/CACATGCGGATCCGCAGTGGctgaagBCACYGCYGNCSCRTA | SEQ ID NO: 18 |
| /5Biosg/CACATGCGGATCCGCAGTGGctgaagAGGGGCTGGARTTCGT | SEQ ID NO: 19 |
| /5Biosg/CACATGCGGATCCGCAGTGGctgaagAGGGGCTGGARTGGGT | SEQ ID NO: 20 |
| /5Biosg/CACATGCGGATCCGCAGTGGctgaagARGGGCTGGARTDSGT | SEQ ID NO: 21 |
| /5Biosg/CACATGCGGATCCGCAGTGGctgaagARGGRYTSGARTDBGT | SEQ ID NO: 22 |
| /5BIOSG/CACATGCGGATCCGCAGTGGCTGAAGTGGCACGGCCCTTCAC | SEQ ID NO: 31 |
| /5BIOSG/CACATGCGGATCCGCAGTGGCTGAAGTGGCACGGCCMTSCAC | SEQ ID NO: 32 |
| /5BIOSG/CACATGCGGATCCGCAGTGGCTGAAGAGGGGCTGGARTTCGT | SEQ ID NO: 33 |
| /5BIOSG/CACATGCGGATCCGCAGTGGCTGAAGAGGGGCTGGARTGGGT | SEQ ID NO: 34 |
| /5BIOSG/CACATGCGGATCCGCAGTGGCTGAAGARGGRYTSGARTWYGT | SEQ ID NO: 35 |
| /5BIOSG/CACATGCGGATCCGCAGTGGCTGAAGARGGRYTSGARTGGGT | SEQ ID NO: 36 |

In some embodiments, the primers of SEQ ID NO:s 15-22 are replaced by SEQ ID NO: 31-36 respectively.

To obtain the chicken CDR2 fragment, a PCR reaction was set up using 2X Phusion MasterMix (NEB), 1 ul cDNA, 0.5 uM forward primer (SEQ ID NO: 14), and 0.5 uM of the reverse primer pool (SEQ ID NOs: 15-18) using the following thermocycling conditions: 98° for 30 sec, 15 cycles of 98° for 5 sec, 65° for 10 sec, 72° for 5 sec, and a final cycle of 72° for 2 min. Following completion of thermocycling, reactions were purified using a PCR purification kit (Qiagen) and captured using 50 ul streptavidin-magnetic beads (NEB). Following capture, beads were washed with wash buffer (0.5 M NaCl, 20 mM Tris-HCl, 0.1 mM EDTA) followed by 1X Cutsmart Buffer (NEB). PCR product was released from the beads by setting up a restriction digestion reaction with AcuI (NEB), Cutsmart Buffer (NEB), and S-adenosylmethionine (NEB) and incubating at 37°, shaking at 1,500 rpm for 30 min in a thermomixer. Following digestion, the supernatant containing released PCR product was purified using a PCR purification kit (Qiagen). This digestion product is a fragment containing chicken CDR2 with overhang at the terminal where CDR2 connects to the FR3 region.

The next step was to ligate this chicken CDR2 fragment to the FR3 human framework region acceptor fragment (SEQ ID NO: 11). A ligation reaction was set up using 1 pmole each of the chicken CDR2 fragment and the human FR3 acceptor fragment, with Quick Ligase Reaction Buffer (NEB) and Quick Ligase (NEB). This reaction was incubated at 25° for 5 min then purified using a PCR purification kit (Qiagen). The next step is a PCR reaction which amplifies the ligation product containing chicken CDR2 and human FR3. The PCR reaction was setup using the purified ligation product as template, 2X Phusion MasterMix (NEB), 0.5 uM reverse primer (SEQ ID NO: 3), and 0.5 uM of the forward primer pool (SEQ ID NOs: 19-22) using the following thermocycling conditions: 98° for 30 sec, 15 cycles of 98° for 5 sec, 61° for 10 sec, 72° for 5 sec, and a final cycle of 72° for 2 min. Following completion of thermocycling, reactions were purified using a PCR purification kit (Qiagen) and captured using 50 ul streptavidin-magnetic beads (NEB). Following capture, beads were washed with wash buffer (0.5 M NaCl, 20 mM Tris-HCl, 0.1 mM EDTA) followed by 1X Cutsmart Buffer (NEB). PCR product was released from the beads by setting up a restriction digestion reaction with AcuI (NEB), Cutsmart Buffer (NEB), and S-adenosylmethionine (NEB) and incubating at 37°, shaking at 1,500 rpm for 30 min in a thermomixer. Following digestion, the supernatant containing released PCR product was purified using a PCR purification kit (Qiagen). The resulting product is a fragment of CDR2-FR3, with an overhang at the terminal where the CDR2 connects to FR2.

The next step was to ligate this CDR2-FR3 fragment to the FR1-CDR1-FR2 human framework region acceptor fragment (SEQ ID NO: 10). A ligation reaction was set up using 1 pmole each of the CDR2-FR3 fragment and the FR1-CDR1-FR2 fragment, with Quick Ligase Reaction Buffer (NEB) and Quick Ligase (NEB). This reaction was incubated at 25° for 5 min then purified using a PCR purification kit (Qiagen). The purified ligation product (a FR1-CDR1-FR2-CDR2-FR3 fragment) is used as template for a final PCR amplification using 2X Phusion MasterMix (NEB), 0.5 uM each primer (SEQ IDs 2-3) using the following thermocycling conditions: 98° for 30 sec, 15 cycles of 98° for 5 sec, 72° for 15 sec, and a final cycle of 72° for 2 min. The PCR product was analyzed by gel electrophoresis and the assembled fragment was excised from the gel and purified using a gel extraction kit (Qiagen). The final product contains chicken VH CDR2 flanked by human VH3-23 FR1-CDR1-FR2 and FR3.

Obtaining the CDR3 region of a chicken antibody VH chain and insertion into the human framework. Chicken VH CDR3 was amplified from cDNA prepared from RNA extracted from B cells of an immunized chicken. The CDR3 is extracted by several PCR and digestion reactions. The CDR3 fragment was then ligated onto the human framework region acceptor fragments, generating a fragment containing chicken CDR3 flanked by human framework regions. The steps are shown in FIG. 2.

The primers used for CDR3 amplification are listed in Table 5 (SEQ ID NOs: 23-30). For each PCR step the primer set includes a pool of primers that bind to chicken framework regions adjacent to the CDR. These pools of primers contain degenerate bases in order to sample as much of the chicken immune repertoire as possible. These primers contain an AcuI restriction enzyme recognition site (ctgaag in Table 5) positioned such that the cleavage of the restriction enzyme leaves an overhang at the desired junction point for ligation to the human framework region acceptor fragments. These primers were biotinylated on the 5′ end for capture onto streptavidin magnetic beads and release by digestion with AcuI. A random spacer sequence between the biotin and AcuI recognition site is included to improve digestion efficiency.

TABLE 5

Primer Sequences for Chicken VH CDR3 Amplification

| | |
|---|---|
| /5Biosg/CACATGCGGATCCGCAGTGGctgaagCCGSCACCTACTACTG | SEQ ID NO: 23 |
| /5Biosg/CACATGCGGATCCGCAGTGGctgaagCCGSCAYCTACTACTG | SEQ ID NO: 24 |
| /5Biosg/CACATGCGGATCCGCAGTGGctgaagCCGSCACCTACTWCTG | SEQ ID NO: 25 |
| /5Biosg/CACATGCGGATCCGCAGTGGctgaagCCGSCAYCTACTWCTG | SEQ ID NO: 26 |
| /5Biosg/CACATGCGGATCCGCAGTGGctgaagCCGSCAYCTAYTWYTG | SEQ ID NO: 27 |
| GGAGGAGACGATGACTTCGGTCC | SEQ ID NO: 28 |
| /5Biosg/CACATGCGGATCCGCAGTGGctgaagCTTCGGTCCCGTGGCC | SEQ ID NO: 29 |
| /5Biosg/CACATGCGGATCCGCAGTGGctgaagCTTCGGTCCCGCGGCC | SEQ ID NO: 30 |
| /5BIOSG/CACATGCGGATCCGCAGTGGCTGAAGCCGSCACCTACTACTG | SEQ ID NO: 37 |
| /5BIOSG/CACATGCGGATCCGCAGTGGCTGAAGCCGSCATCTACTACTG | SEQ ID NO: 38 |
| /5BIOSG/CACATGCGGATCCGCAGTGGCTGAAGCCGSCACCTACTTCTG | SEQ ID NO: 39 |
| /5BIOSG/CACATGCGGATCCGCAGTGGCTGAAGCCGSCATCTACTTCTG | SEQ ID NO: 40 |
| /5BIOSG/CACATGCGGATCCGCAGTGGCTGAAGCCGSCAYCTACTWYTG | SEQ ID NO: 41 |
| GGAGGAGACGGTGACTAGGGTCCCCTGGCCCCA | SEQ ID NO: 42 |

To obtain the chicken CDR3 fragment, a PCR reaction was set up using 2X Phusion MasterMix (NEB), 1 ul cDNA, 0.5 uM forward primer pool (SEQ ID NOs: 23-27), and 0.5 uM of the reverse primer (SEQ ID NO: 28) using the following thermocycling conditions: 98° for 30 sec, 15 cycles of 98° for 5 sec, 52° for 10 sec, 72° for 5 sec, and a final cycle of 72° for 2 min. Following completion of thermocycling, reactions were purified using a PCR purification kit (Qiagen) and captured using 50 ul streptavidin-magnetic beads (NEB). Following capture, beads were washed with wash buffer (0.5 M NaCl, 20 mM Tris-HCl, 0.1 mM EDTA) followed by 1X Cutsmart Buffer (NEB). PCR product was released from the beads by setting up a restriction digestion reaction with AcuI (NEB), Cutsmart Buffer (NEB), and S-adenosylmethionine (NEB) and incubating at 37°, shaking at 1,500 rpm for 30 min in a thermomixer. Following digestion, the supernatant containing released PCR product was purified using a PCR purification kit (Qiagen). The resulting product a fragment containing chicken CDR3 with overhang at the terminal where CDR3 connects to the FR3 region.

This product was ligated to the FR3 human framework region acceptor fragment (SEQ ID NO: 12) by setting up a ligation reaction containing 1 pmole each of the digested PCR product and acceptor fragment, Quick Ligase Reaction Buffer (NEB), and Quick Ligase (NEB). This reaction was incubated at 25° for 5 min then purified using a PCR purification kit (Qiagen). To amplify the ligation product, the next PCR step was setup using the purified ligation reaction as template, 2X Phusion MasterMix (NEB), 0.5 uM forward primer (SEQ ID NO: 6), and 0.5 uM of the reverse primer pool (SEQ IDs 29-30) using the following thermocycling conditions: 98° for 30 sec, 15 cycles of 98° for 5 sec, 68° for 10 sec, 72° for 5 sec, and a final cycle of 72° for 2 min. Following completion of thermocycling, reactions were purified using a PCR purification kit (Qiagen) and captured using 50 ul streptavidin-magnetic beads (NEB). Following capture, beads were washed with wash buffer (0.5 M NaCl, 20 mM Tris-HCl, 0.1 mM EDTA) followed by 1X Cutsmart Buffer (NEB). PCR product was released from the beads by setting up a restriction digestion reaction with AcuI (NEB), Cutsmart Buffer (NEB), and S-adenosylmethionine (NEB) and incubating at 37°, shaking at 1,500 rpm for 30 min in a thermomixer. Following digestion, the supernatant containing released PCR product was purified using a PCR purification kit (Qiagen). The resulting product is a fragment of FR3-CDR3, with an overhang at the terminal where the CDR3 connects to FR4.

This product was ligated to the FR4 acceptor fragment (SEQ ID NO: 13) by setting up a ligation reaction containing 1 pmole each of the digested PCR product and acceptor fragment, Quick Ligase Reaction Buffer (NEB), and Quick Ligase (NEB). This reaction was incubated at 25° for 5 min then purified using a PCR purification kit (Qiagen). The purified ligation reaction is used as template for a final PCR amplification using 2X Phusion MasterMix (NEB), 0.5 uM each primer (SEQ ID NOs: 6,9) using the following thermocycling conditions: 98° for 30 sec, 15 cycles of 98° for 5 sec, 72° for 15 sec, and a final cycle of 72° for 2 min. The product was analyzed by gel electrophoresis and the assembled fragment was excised from the gel and purified using a gel extraction kit (Qiagen). The final product contains chicken VH CDR3 flanked by human VH3-23 FR3 and FR4.

In some embodiments, the sequences of SEQ ID NO: 37-41 are used in the place of SEQ ID NO: 23-27. In some embodiments, the sequence of SEQ ID NO: 42 is used instead SEQ ID NO: 28. These can be used and are still able to prime with mismatches due to high Tm, which can be used to not include a second PCR/digestion/ligation steps as described herein.

### Assembly of the Humanized Antibody VH Chain

VH was assembled by overlap PCR using the humanized chicken CDR2 and CDR3 fragments (fragments FR1-CDR1-FR2-CDR2-FR3 and FR3-CDR3-FR4). The PCR reaction was set up using 2X Phusion Mastermix (NEB), 0.5 uM each primer (SEQ ID NOs: 2,9), and 1 pmole of each fragment using the following conditions: 98° for 30 sec, 15 cycles of 98° for 5 sec, 72° for 20 sec, and a final cycle of 72° for 2 min. The product was analyzed by gel electrophoresis and the assembled fragment was excised from the gel and purified using a gel extraction kit (Qiagen).

The final product is a humanized VH coding sequence containing human VH3-23 FR1, CDR1, FR2, and FR3 regions, human JH1, with chicken derived CDR2 and CDR3. This is paired with a humanized VL fragment containing chicken CDRs prepared in a similar manner to generate a humanized antibody library. The library is cloned into a phagemid for affinity selection by phage display against the immunized antigen.

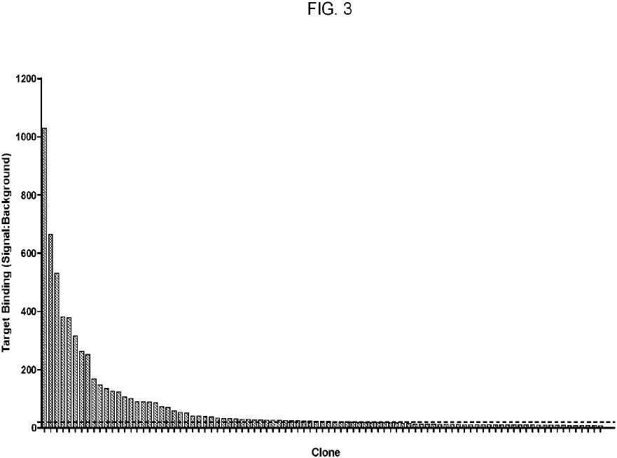

Affinity maturation and binding tests. A humanized scFv library was constructed using CDRs extracted from a chicken immunized as provided herein and described above with a membrane protein antigen and cloned into a phagemid vector. Following two rounds of affinity selection against the target antigen by phage display, periplasm extracts were prepared from bacterial cultures of individual clones. scFv extracts were tested by ELISA. The test results are shown FIG. 3.

These results illustrate that the production of antibodies utilizing the libraries produced herein was successful and can be used to generate a diverse set of antibodies that have specific binding.

What is claimed is:

1-59. (canceled)

60. A method of producing a population of nucleic acid molecules encoding a chicken complementary determining region (CDR) flanked by two human framework regions (FRs), the method comprising:
a) amplifying a first population of nucleic acid molecules encoding chicken antibodies with a first primer and a second primer under conditions sufficient to produce the amplified population of nucleic acid molecules encoding the chicken complementary determining region (CDR), wherein:
the first primer anneals to a region upstream or downstream of a CDR of the chicken antibody, wherein the first primer contains a restriction enzyme recognition site recognized by a restriction enzyme that cleaves at a location that is immediately upstream or downstream of the CDR and at a distance away from the recognition site; and
the second primer anneals to a region at a distance downstream of the CDR of the chicken antibody, if the first primer anneal upstream of the CDR; or
the second primer anneals to a region at a distance upstream of the CDR of the chicken antibody, if the first primer anneals downstream of the CDR.

61. The method of claim 60, further comprising digesting the amplified population of nucleic acid molecules with said restriction enzyme to produce a 5′ overhang immediately upstream of the sequence encoding the CDR to produce a digestion product.

62. The method of claim 61, further comprising preparing a first ligation product by ligating said digestion product to a nucleic acid sequence encoding a first FR of a human antibody,
wherein said first FR of a human antibody comprises an overhang region at its 3′-end compatible with the overhang region of said digestion product so that the first FR is ligated to the upstream of the first digestion product.

63. The method of claim 60, wherein the restriction enzyme cleaves at a location that is 1, 2, 3, 4, or 5 nucleotides of the CDR boundary, wherein the boundary is either internal or external of the sequencing encoding the CDR.

64. The method of claim 63, wherein the restriction enzyme cleaves at a location that is 1, 2, or 3 nucleotides of the CDR boundary.

65. The method of claim 60, wherein the restriction enzyme cleaves at a location that is at least 10 nucleotides away from the recognition site.

66. The method of claim 60, wherein the second primer anneals to a region at a distance no greater than the length of the mRNA transcript downstream or upstream of the CDR of the chicken antibody.

67. The method of claim 61, wherein the 5′ overhang that is immediately downstream is 1, 2, 3, 4, or 5 nucleotides of the CDR boundary.

68. The method of claim 62, the method further comprising preparing a second population of amplified nucleic acid molecules with a third primer and a fourth primer under conditions sufficient to produce the second amplified population of nucleic acid molecules encoding the chicken CDR, wherein:
the third primer anneals to a region immediately downstream of the nucleic acid sequence encoding the CDR present in the first ligation product, wherein the third primer contains a restriction enzyme recognition site recognized by a restriction enzyme that cleaves at a location that is immediately downstream of the CDR and at a distance away from the recognition site; and
the fourth primer anneals to a portion of the nucleic acid molecule encoding the first FR present in the first ligation product at a distance upstream of the CDR in the ligation product.

69. The method of claim 68, wherein the second primer anneals to a region at a distance no greater than the length of the mRNA transcript downstream of the CDR of the chicken antibody.

70. The method of claim 68, further comprising digesting the second amplified population of nucleic acid molecules with said restriction enzyme to produce a 5′ overhang immediately downstream of the sequence encoding the CDR to produce a second digestion product.

71. The method of claim 70, further comprising ligating the second digestion product to a nucleic acid sequence encoding a second FR of a human antibody to produce the second ligation product, wherein said second FR comprises an overhang region at its 5′-end compatible with the overhang region of said second digestion product, so that the second FR is ligated to the downstream of the second digestion product.

72. The method of claim 70, further comprising amplifying the second ligation product with a first FW primer that anneals to the first FR of the human antibody and a second primer that anneals to the second FR of the human antibody under conditions sufficient to produce the population of nucleic acid molecules encoding the CDR flanked by two FRs of a human antibody.

73. The method of claim 60, wherein the CDR is a chicken CDR1, CDR2, or CDR3 and
the FRs are human FR1, FR2, FR3, or FR4.

74. The method of claim 60, wherein the restriction enzyme is a Type IIS enzyme.

75. The method of claim 60, wherein the first primer and third primer comprise a nucleic acid sequence comprising the formula: 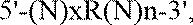
wherein,
R is the recognition sequence;
N is any nucleic acid base, such as naturally occurring, non-naturally occurring, or degenerate nucleic acid base;
x is 0-11; and
n is 1-21.

76. The method of claim 75, wherein R is CTGAAG, CGATC, ACNNNNGTAYC, GAAGAC, GCAGC, CCATC, ACGGC, CGANNNNNNTGC, GTATCC, GTCTC, ACCTGC, ACTGGG, CTGGAG, CTTGAG, GGTCTC, ACNNNNNCTCC, GAGGAG, GTGCAG, GTCTC, CGTCTC, GGGAC, GAATGC, CTCAG, ACCTGC, GCTCTTC, GCAATG, ACTGG, GCGATG, GGATG, GCAGTG, CAGTG, CAANNNNNGTGG, CTCTTC, GGCGGA, CGTCTC, CCCGC, GGATG, GACGC, GGTGA, CCTTC, GAAGA, GAGTC, TCCRAC, CCTC, GCCGAG, GAGTC, GCTCTTC, or GCATC.

77. The method of claim 60, wherein the first primer and third primer comprise:
a nucleic acid sequence comprises a sequence of 5′-CTGAAGNNNNNNNNNNNNNNNN-3′ (SEQ ID NO: 45), wherein N is any nucleic acid base, such as naturally occurring, non-naturally occurring, or degenerate nucleic acid base;
a sequence of 5′-CTGGAGNNNNNNNNNNNNNNNN-3′, wherein N is any nucleic acid base, such as naturally occurring, non-naturally occurring, or degenerate nucleic acid base;
a nucleic acid sequence comprises a sequence of 5′-CTTGAGNNNNNNNNNNNNNNNN-3′, wherein N is any nucleic acid base, such as naturally occurring, non-naturally occurring, or degenerate nucleic acid base;
a sequence of 5′-GTGCAGNNNNNNNNNNNNNNNN-3′ (SEQ ID NO: 62), wherein N is any nucleic acid base, such as naturally occurring, non-naturally occurring, or degenerate nucleic acid base;
a nucleic acid sequence comprises a sequence of 5′-TCCRACNNNNNNNNNNNNNNNNNNNN-3′, wherein N is any nucleic acid base, such as naturally occurring, non-naturally occurring, or degenerate nucleic acid base; or
a nucleic acid sequence comprises a sequence of 5′-GCCGAGNNNNNNNNNNNNNNNNNNNNN-3′, wherein N is any nucleic acid base, such as naturally occurring, non-naturally occurring, or degenerate nucleic acid base.

78. A method of producing a library of nucleic acid molecules encoding humanized variable regions of antibodies, the method comprising combining:
i) a first library of nucleic acid molecules encoding a chicken complementary determining region 1 (CDR1) domain, flanked by nucleic acid sequences encoding a human framework region 1 (FR1) and a human framework region 2 (FR2);
ii) a second library of nucleic acid sequences encoding a chicken complementary determining region 2 (CDR2) domain, flanked by nucleic acid sequences encoding a human framework region 2 (FR2) and a human framework region 3 (FR3);
iii) a third library of nucleic acid sequences encoding a chicken complementary determining region 3 (CDR3) domain, flanked by nucleic acid sequences encoding a human framework region 3 (FR3) and a human framework region 4 (FR4),
wherein the nucleic acid molecule encoding the humanized variable regions of antibodies has a formula of: 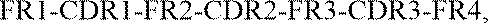
wherein
FR1 is a human FR1;
CDR1 is a chicken CDR1;
FR2 is a human FR2;
CDR2 is a chicken CDR2;
FR3 is a human FR3;
CDR3 is a chicken CDR3; and
FR4 is a human FR4.

79. An oligonucleotide that anneals to a region immediately upstream or downstream of CDR of a chicken antibody, and wherein said oligonucleotide comprises a restriction enzyme recognition site recognized by a restriction enzyme that cleaves at a distance downstream of the recognition site, wherein the oligonucleotide comprises the sequence of nucleic acid sequence comprising the formula: 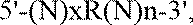
wherein,
R is the recognition sequence;
N is any nucleic acid base, such as naturally occurring, non-naturally occurring, or degenerate nucleic acid base;
x is 0-11; and
n is 1-21.
